(19) United States
(12) Patent Application Publication
(10) Pub. No.: US 2010/0317679 A1
(43) Pub. Date: Dec. 16, 2010

(54) SUBSTITUTED ARYL-FUSED SPIROCYCLIC AMINES

(75) Inventors: Yuelian Xu, Newington, CT (US); Linghong Xie, Southborough, MA (US)

Correspondence Address: CANTOR COLBURN LLP, 20 Church Street, 22nd Floor, Hartford, CT, 06103, US

(73) Assignee: LIGAND PHARMACEUTICALS, INC., La Jolla, CA (US)

(21) Appl. No.: 12/678,782

(22) PCT Filed: Sep. 19, 2008
(86) PCT No.: PCT/US2008/077103

§ 371 Date: Aug. 30, 2010

Related U.S. Application Data
(60) Provisional application No. 60/974,108, filed on Sep. 21, 2007.

(51) Int. Cl. A61K 31/506; C07D 401/10; A61K 31/438; A61P 25/18; A61P 25/28; A61P 25/06; A61P 25/08; A61P 3/10; A61P 3/04; A61P 25/16; G01N 33/68; C07D 401/12

(52) U.S. Cl. 514/275; 546/17; 514/278; 436/86; 544/230; 436/57

(57) ABSTRACT

Substituted Aryl-fused Spirocyclic Amines of the formula (I): are provided, as are methods for their preparation and use. Such compounds may generally be used to modulate ligand binding to histamine H3 receptors in vivo or in vitro, and are particularly useful in the treatment of a variety of disorders in humans, domesticated companion animals and livestock animals. Pharmaceutical compositions and therapeutic methods are provided, as are methods for using such ligands for detecting histamine H3 receptors (e.g., receptor localization studies).

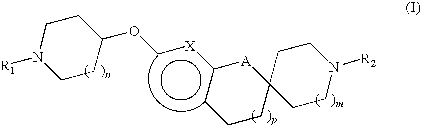

### FIELD OF THE INVENTION

This invention relates generally to substituted aryl-fused spirocyclic amines, and to the use of such compounds for treating conditions responsive to histamine H3 receptor modulation. The invention further relates to the use of such compounds as probes for the detection and localization of histamine H3 receptors.

### BACKGROUND OF THE INVENTION

Hormones and neurotransmitters regulate a wide variety of biological functions, often via specific receptor proteins located on the surface of living cells. Many of these receptors carry out intracellular signaling via the activation of coupled guanosine triphosphate-binding proteins (G proteins); such receptors are collectively called G protein-coupled receptors or GPCRs. The important role of GPCRs in the regulation of cell and organ function has attracted attention to these receptors as targets for new pharmaceutical agents.

Histamine is a multifunctional chemical transmitter that signals through specific cell surface GPCRs. To date, four histamine receptor subtypes have been identified: H1, H2, H3 and H4. Histamine H3 receptor is a presynaptic GPCR that is found primarily in the central nervous system, although lower levels are also found in the peripheral nervous system. Genes encoding the H3 receptor have been reported in various organisms, including humans (see Lovenberg et al. (1999) Molecular Pharmacology 55:1101-07), and alternative splicing of this gene appears to result in multiple isoforms. The histamine H3 receptor is an auto- and hetero-receptor whose activation leads to a decreased release of neurotransmitters (including histamine, acetylcholine, norepinephrine and glutamate) from neurons in the brain. Histamine H3 receptor is involved in the regulation of processes such as sleep and wakefulness, feeding and memory.

Antagonists of histamine H3 receptor increase synthesis and release of cerebral histamine and other neurotransmitters, inducing an extended wakefulness, an improvement in cognitive processes, a reduction in food intake and a normalization of vestibular reflexes. Such antagonists are useful, for example, as therapeutics for central nervous system disorders such as Alzheimer's disease, Parkinson's disease, schizophrenia, mood and attention alterations including attention deficit hyperactivity disorder and attention deficit disorder, memory and learning disorders, cognitive disorders (such as mild cognitive impairment and cognitive deficits in psychiatric pathologies), epilepsy, migraine, and disorders associated with the regulation of sleep and wakefulness, as well as in the treatment and prevention of conditions such as obesity, eating disorders, diabetes, vertigo, motion sickness and allergic rhinitis.

Accordingly, there is a need for new H3 receptor modulators. The present invention fulfills this need, and provides further related advantages.

### SUMMARY OF THE INVENTION

In certain aspects, the present invention provides substituted aryl-fused spirocyclic amines of Formula I:

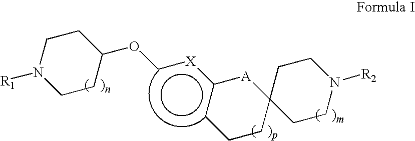

or a pharmaceutically acceptable salt or hydrate thereof, wherein:
A is CH₂ or O;
X is CH or N, such that

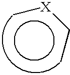

represents a 5- or 6-membered heteroaryl that is fused to the ring represented by

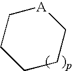

and is optionally substituted (e.g., with C₁-C₆alkyl or (C₃-C₈cycloalkyl)C₁-C₄alkyl);
n, m and p are independently 0, 1 or 2;
R₁ is C₁-C₆alkyl or (C₃-C₈cycloalkyl)C₀-C₂alkyl, each of which is substituted with from 0 to 4 substituents independently chosen from amino, halogen, cyano, hydroxy, nitro, oxo, aminocarbonyl, aminosulfonyl, —COOH, C₁-C₆alkyl, C₂-C₆alkenyl, C₂-C₆alkynyl, C₁-C₆alkoxy, C₁-C₆haloalkyl, C₁-C₆haloalkoxy, C₂-C₆alkyl ether, C₁-C₆alkanoyl, C₃- C₆alkanone, C₁-C₆alkoxycarbonyl, C₁-C₆alkylsulfonyl, mono- or di-(C₁-C₆alkyl)aminocarbonyl, or mono-or di-(C₁-C₆alkyl)aminoC₀-C₄alkyl; and
R₂ is C₁-C₆alkyl, C₂-C₆alkenyl, C₂-C₆alkynyl, (C₃-C₈cycloalkyl)C₀-C₂alkyl, C₁-C₆haloalkyl, C₁-C₆alkanoyl, C₁-C₆alkylsulfonyl, mono- or di-(C₁-C₆alkyl)aminocarbonyl, mono- or di-(C₁-C₆alkyl)aminosulfonyl, phenylC₀-C₂alkyl, (4- to 8-membered heterocycle)C₀-C₂alkyl; each of which is substituted with from 0 to 4 substituents independently chosen from amino, halogen, cyano, hydroxy, nitro, oxo, aminocarbonyl, aminosulfonyl, —COOH, C₁-C₆alkyl, C₂-C₆alkenyl, C₂-C₆alkynyl, C₁-C₆alkoxy, C₁-C₆haloalkoxy, C₂-C₆alkyl ether, C₁-C₆alkanoyl, C₁-C₆alkylsulfonyl, mono- or di-(C₁-C₆alkyl)amino, mono- or di-(C₁-C₆alkyl)aminocarbonyl, mono- or di-(C₁-C₆alkyl)aminosulfonyl, phenyl and 5- or 6-membered heteroaryl.

Within certain aspects, substituted aryl-fused spirocyclic amines provided herein are H3 receptor modulators that exhibit a Kᵢ at a histamine H3 receptor, preferably a human H3 receptor, that is no greater than 4 micromolar, 1 micromolar, 500 nanomolar, 100 nanomolar, 50 nanomolar or 10 nanomolar, as determined using an assay for H3 receptor GTP binding.

Within certain aspects, substituted aryl-fused spirocyclic amines provided herein are labeled with a detectable marker (e.g., radiolabeled or fluorescein conjugated).

The present invention further provides, within other aspects, pharmaceutical compositions comprising at least one substituted aryl-fused spirocyclic amine as provided herein in combination with a physiologically acceptable carrier or excipient.

Within further aspects, methods are provided for modulating H3 receptor activity, comprising contacting a cell (e.g., neuronal) expressing H3 receptor with at least one H3 receptor modulator as described herein. Such contact may occur in vivo or in vitro and is generally performed using a concentration of compound that is sufficient to alter H3 receptor GTP binding in vitro (e.g., using the assay provided in Example 7, herein).

The present invention further provides methods for treating a condition responsive to H3 receptor modulation in a patient, comprising administering to the patient a therapeutically effective amount of at least one H3 receptor modulator. Such conditions include, for example, attention deficit disorder, attention deficit hyperactivity disorder, dementia, schizophrenia, cognitive disorders (including mild cognitive impairment), epilepsy, migraine, excessive daytime sleepiness (EDS) and related disorders such as shift work disorder, fatigue and fatigue-related disorders, jet lag, narcolepsy, sleep apnea, allergic rhinitis, vertigo, motion sickness, memory disorders such as Alzheimer's disease, Parkinson's disease, obesity, eating disorders and diabetes.

Within further aspects, the present invention provides methods for determining the presence or absence of H3 receptor in a sample, comprising: (a) contacting a sample with a H3 receptor modulator as described herein under conditions that permit binding of the H3 receptor modulator to H3 receptor; and (b) detecting a level of the H3 modulator bound to H3 receptor.

The present invention also provides packaged pharmaceutical preparations, comprising: (a) a pharmaceutical composition as described herein in a container; and (b) instructions for using the composition to treat one or more conditions responsive to H3 receptor modulation, such as the conditions recited herein.

In yet another aspect, the present invention provides methods of preparing the compounds disclosed herein, including the intermediates.

These and other aspects of the present invention will become apparent upon reference to the following detailed description.

### DETAILED DESCRIPTION

As noted above, the present invention provides substituted aryl-fused spirocyclic amines. Such compounds may be used in vitro or in vivo, to modulate H3 receptor activity in a variety of contexts.

### Terminology

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that includes variables (e.g., R₁, Z, etc.). Unless otherwise specified, each variable within such a formula is defined independently of any other variable, and any variable that occurs more than one time in a formula is defined independently at each occurrence.

The phrase “substituted aryl-fused spirocyclic amines,” as used herein, encompasses all compounds of Formula I, including any enantiomers, racemates and stereoisomers, as well as pharmaceutically acceptable salts, solvates (e.g., hydrates) and esters of such compounds.

A “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutically acceptable anions for use in salt formation include, but are not limited to, acetate, 2-acetoxybenzoate, ascorbate, benzoate, bicarbonate, bromide, calcium edetate, carbonate, chloride, citrate, dihydrochloride, diphosphate, ditartrate, edetate, estolate (ethylsuccinate), formate, fumarate, gluceptate, gluconate, glutamate, glycolate, glycollylarsanilate, hexylresorcinate, hydrabamine, hydrobromide, hydrochloride, hydroiodide, hydroxymaleate, hydroxynaphthoate, iodide, isethionate, lactate, lactobionate, malate, maleate, mandelate, methylbromide, methylnitrate, methylsulfate, mucate, napsylate, nitrate, pamoate, pantothenate, phenylacetate, phosphate, polygalacturonate, propionate, salicylate, stearate, subacetate, succinate, sulfamate, sulfanilate, sulfate, sulfonates including besylate (benzenesulfonate), camsylate (camphorsulfonate), edisylate (ethane-1,2-disulfonate), esylate (ethanesulfonate) 2-hydroxyethylsulfonate, mesylate (methanesulfonate), triflate (trifluoromethanesulfonate) and tosylate (p-toluenesulfonate), tannate, tartrate, teoclate and triethiodide. Similarly, pharmaceutically acceptable cations for use in salt formation include, but are not limited to ammonium, benzathine, chloroprocaine, choline, diethanolamine, ethylenediamine, meglumine, procaine, and metals such as aluminum, calcium, lithium, magnesium, potassium, sodium and zinc. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, methanol, isopropanol or acetonitrile, is preferred.

It will be apparent that each compound provided herein may, but need not, be formulated as a solvate (e.g., hydrate), ester or non-covalent complex. In addition, the various crystal forms and polymorphs are within the scope of the present invention. Also provided herein are prodrugs of the compounds of the recited Formulas. A “prodrug” is a compound that may not fully satisfy the structural requirements of the compounds provided herein, but is modified in vivo, following administration to a patient, to produce a compound a formula provided herein. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxy, amino, or sulfhydryl group, respectively. Examples of prodrugs include, but are not limited to, esters such as acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

As used herein, the term “alkyl” refers to a straight or branched chain saturated aliphatic hydrocarbon. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl and 3-methylpentyl.

The term “alkylene” refers to a divalent alkyl group, which may be straight or branched. C₁-C₄alkylene is an alkylene group having from 1 to 4 carbon atoms. “C₀-C₄alkyl” or “C₀-C₄alkylene” is a single covalent bond (C₀) or an alkylene group having from 1 to 4 carbon atoms.

“Alkenyl” refers to straight or branched chain alkene groups, which comprise at least one unsaturated carbon-carbon double bond. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

A “cycloalkyl” is a group that comprises one or more saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Cycloalkyl groups do not comprise an aromatic ring or a heterocyclic ring. A “(C₃-C₈cycloalkyl)C₀-C₂alkyl” is a C₃-C₈cycloalkyl group linked via a single covalent bond or a methylene or ethylene group; C₃-C₇cycloalkyl is a directly linked 3- to 7-membered cycloalkyl.

By “alkoxy,” as used herein, is meant an alkyl group attached via an oxygen bridge. “C₁-C₆alkoxy” has from 1 to 6 carbon atoms in the alkyl portion of the group. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups. Similarly, “alkylthio” refers to an alkyl group attached via a sulfur bridge.

The term “oxo” is used herein to refer to an oxygen substituent of a carbon atom that results in the formation of a carbonyl group (C═O). An oxo group that is a substituent of a nonaromatic carbon atom results in a conversion of —CH₂— to —C(═O)—. An oxo group that is a substituent of an aromatic carbon atom results in a conversion of —CH— to —C(═O)— and may result in a loss of aromaticity.

The term “alkanoyl” refers to an acyl group (e.g., —(C═O)-alkyl), in which carbon atoms are in a linear or branched alkyl arrangement and where attachment is through the carbon of the keto group. Alkanoyl groups have the indicated number of carbon atoms, with the carbon of the keto group being included in the numbered carbon atoms. For example a C₂alkanoyl group is an acetyl group having the formula —(C═O)CH₃; “C₁alkanoyl” refers to —(C═O)H. “C₁-C₆alkanoyl groups” contain from 1 to 6 carbon atoms.

An “alkanone” is a ketone group in which carbon atoms are in a linear or branched alkyl arrangement. “C₃-C₆alkanone” refers to an alkanone having from 3 to 6 carbon atoms, respectively. By way of example, a C₃ alkanone group has the structure —CH₂—(C═O)—CH₃.

Similarly, “alkyl ether” refers to a linear or branched ether substituent (i.e., an alkyl group that is substituted with an alkoxy group). A C₂ alkyl ether has the structure —CH₂—O—CH₃; A C₂-C₆alkyl ether has a total of 2, 3, 4, 5 or 6 carbon atoms.

The term “alkoxycarbonyl” refers to an alkoxy group attached through a keto (—(C═O)—) bridge (i.e., an alkoxycarbonyl group has the general structure —C(═O)—O-alkyl). “C₁alkoxycarbonyl” refers to —C(═O)—O—CH₃; C₃alkoxycarbonyl indicates —C(═O)—O—(CH₂)₂CH₃ or —C(═O)—O—(CH)(CH₃)₂ (i.e., the carbon of the keto bridge is not included in the indicated number of carbon atoms). “C₁-C₆alkoxycarbonyl” groups have from 1 to 6 carbon atoms in the alkyl portion of the group.

“Alkylsulfonyl” refers to groups of the formula —(SO₂)-alkyl, in which the sulfur atom is the point of attachment. “C₁-C₆alkylsulfonyl” has from 1 to 6 carbon atoms in the alkyl group.

The term “aminocarbonyl” refers to an amide group (i.e., —(C═O)NH₂). The term “mono- or di-(C₁-C₆alkyl)aminocarbonyl” refers to groups of the formula —(C═O)—N(R)₂, in which the carbonyl is the point of attachment, one R is C₁-C₆alkyl and the other R is hydrogen or an independently chosen C₁-C₆alkyl.

“Alkylamino” refers to a secondary or tertiary amine that has the general structure —NH-alkyl or —N(alkyl)(alkyl), wherein each alkyl is selected independently from alkyl, cycloalkyl and (cycloalkyl)alkyl groups. Such groups include, for example, mono- and di-(C₁-C₆alkyl)amino groups, in which each C₁-C₆alkyl may be the same or different.

“Alkylaminoalkyl” refers to an alkylamino group linked via an alkylene group (i.e., a group having the general structure -alkylene-NH-alkyl or -alkylene-N(alkyl)(alkyl)) in which each alkyl is selected independently from alkyl, cycloalkyl and (cycloalkyl)alkyl groups. Alkylaminoalkyl groups include, for example, mono- and di-(C₁-C₆alkyl)aminoC₁-C₄alkyl. “Mono- or di-(C₁-C₆alkyl)aminoC₀-C₄alkyl” refers to a mono- or di-(C₁-C₆alkyl)amino group linked via a single covalent bond or a C1-C4alkylene group (e.g., methylene or ethylene). The following are representative alkylaminoalkyl groups:

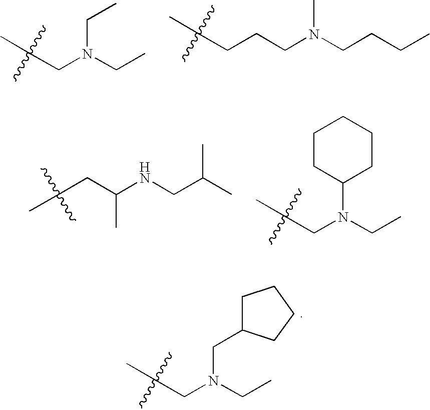

It will be apparent that the definition of “alkyl” as used in the terms “alkylamino” and “alkylaminoalkyl” differs from the definition of “alkyl” used for all other alkyl-containing groups, in the inclusion of cycloalkyl and (cycloalkyl)alkyl groups (e.g., (C₃-C₇cycloalkyl)C₀-C₆alkyl).

The term “halogen” refers to fluorine, chlorine, bromine or iodine.

A “haloalkyl” is an alkyl group that is substituted with 1 or more halogen atoms (e.g., “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “C₁-C₆haloalkoxy” groups have 1 to 6 carbon atoms.

A dash (“—”) that is not between two letters or symbols is used to indicate a point of attachment for a substituent. For example, —CONH₂ is attached through the carbon atom.

A “carbocycle” or “carbocyclic group” comprises at least one ring formed entirely by carbon-carbon bonds (referred to herein as a carbocyclic ring), and does not contain a heterocycle. Certain representative carbocycles are cycloalkyl as described above. Other carbocycles are aryl (i.e., contain at least one aromatic ring). PhenylC₀-C₂alkyl is a phenyl, benzyl or phenethyl moiety.

A “heterocycle” or “heterocyclic group” has from 1 to 3 fused, pendant or spiro rings (and typically from 3 to 15 ring members in total), at least one of which is a heterocyclic ring (i.e., one or more ring atoms is a heteroatom independently chosen from O, S and N, with the remaining ring atoms being carbon). Additional rings, if present, may be heterocyclic or carbocyclic. Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and certain heterocycles comprising fused, pendant or spiro rings contain from 9 to 14 ring members. Certain heterocycles comprise a sulfur atom as a ring member; in certain embodiments, the sulfur atom is oxidized to SO or SO₂. Heterocycles may be optionally substituted with a variety of substituents, as indicated. Unless otherwise specified, a heterocycle may be a heterocycloalkyl group (i.e., each ring is saturated or partially saturated) or a heteroaryl group (i.e., at least one ring within the group is aromatic), and may be linked via any ring atom, provided that a stable compound results.

Heterocyclic groups include, for example, acridinyl, azepanyl, azocinyl, benzimidazolyl, benzimidazolinyl, benzisothiazolyl, benzisoxazolyl, benzofuranyl, benzothiofuranyl, benzothiophenyl, benzoxazolyl, benzothiazolyl, benzotriazolylcarbazolyl, benztetrazolyl, NH-carbazolyl, carbolinyl, chromanyl, chromenyl, cinnolinyl, decahydroquinolinyl, dihydrofuro[2,3-b]tetrahydrofuran, dihydroisoquinolinyl, dihydrotetrahydrofuranyl, 1,4-dioxa-8-aza-spiro[4.5]dec-8-yl, dithiazinyl, furanyl, furazanyl, imidazolinyl, imidazolidinyl, imidazolyl, indazolyl, indolenyl, indolinyl, indolizinyl, indolyl, isobenzofuranyl, isochromanyl, isoindazolyl, isoindolinyl, isoindolyl, isothiazolyl, isoxazolyl, isoquinolinyl, morpholinyl, naphthyridinyl, octahydroisoquinolinyl, oxadiazolyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, piperidinyl, piperidonyl, pteridinyl, purinyl, pyranyl, pyrazinyl, pyrazolidinyl, pyrazolinyl, pyrazolyl, pyridazinyl, pyridoimidazolyl, pyridooxazolyl, pyridothiazolyl, pyridyl, pyrimidyl, pyrrolidinyl, pyrrolidonyl, pyrrolinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienothiazolyl, thienooxazolyl, thienoimidazolyl, thienyl, thiophenyl, thiomorpholinyl and variants thereof in which the sulfur atom is oxidized, triazinyl, xanthenyl and any of the foregoing that are substituted as described herein.

Certain heterocycles are 5- or 6-membered heteroaryl groups (e.g., pyridyl, pyrimidyl and pyridazinyl), each of which may be substituted as indicated. Other heterocycles are 4- to 8-membered heterocycloalkyl groups, which are saturated or partially saturated heterocycles as described above, containing 4, 5, 6, 7 or 8 ring members. A “(4- to 7-membered heterocycloalkyl)C₀-C₂alkyl” is a 4- to 7-membered heterocycloalkyl group that is linked via a single covalent bond or a methylene or ethylene group.

A “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member. The term “substitution” refers to replacing one or more hydrogen atoms in a molecular structure with a substituent as described above, such that the valence on the designated atom is not exceeded, and such that a chemically stable compound (i.e., a compound that can be isolated, characterized, and tested for biological activity) results from the substitution.

Groups that are “optionally substituted” are unsubstituted or substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of permissible substituents. Certain optionally substituted groups are substituted with from 0 to 2, 3 or 4 independently selected substituents (i.e., are unsubstituted or substituted with up to the recited maximum number of substituents). Other optionally substituted groups are substituted with at least one substituent (e.g., substituted with from 1 to 2, 3 or 4 independently selected substituents).

Unless otherwise specified, the term “H3 receptor” is used herein to refer to any histamine H3 subtype receptor, including human H3 receptor (see, e.g., U.S. Pat. No. 6,136,559), H3 receptor found in other mammals and chimeric receptors retaining H3 function, including the chimeric H3 receptor provided as SEQ ID NO:8 in U.S. patent application Ser. No. 11/355,711, which published as US 2006/0188960.

A “H3 receptor modulator,” also referred to herein as a “modulator,” is a compound that modulates H3 receptor GTP binding. A H3 receptor modulator may be a H3 receptor agonist or antagonist. A modulator binds with “high affmity” if the K, at H3 receptor is less than 4 micromolar, preferably less than 1 micromolar, 500 nanomolar, 100 nanomolar, 50 nanomolar or 10 nanomolar. A representative assay for evaluating an effect on H3 receptor GTP binding is provided in Example 7, herein.

Unless otherwise specified, the terms “IC₅₀” and “EC₅₀,” as used herein, refer to values obtained using the assay as described in Example 7.

A modulator is considered an “antagonist” if it detectably inhibits H3 receptor agonist-stimulated GTP binding (using, for example, the representative assay provided in Example 7); in general, such an antagonist inhibits such GTP binding with a IC₅₀ value of less than 4 micromolar, preferably less than 1 micromolar, 500 nanomolar, 100 nanomolar, 50 nanomolar or 10 nanomolar. H3 receptor antagonists include neutral antagonists and inverse agonists.

An “inverse agonist” of H3 receptor is a compound that reduces the GTP binding activity of H3 receptor below its basal activity level in the absence of added agonist. Inverse agonists of H3 receptor may also inhibit the activity in the presence of agonist. The basal activity of H3 receptor, as well as the reduction in H3 receptor GTP binding activity due to the presence of H3 receptor antagonist, may be determined using the assay of Example 7.

A “neutral antagonist” of H3 receptor is a compound that inhibits the activity of H3 receptor agonist, but does not significantly change the basal activity of the receptor (i.e., within the assay of Example 7 performed in the absence of agonist, H3 receptor activity is reduced by no more than 10%, preferably by no more than 5%, and more preferably by no more than 2%; most preferably, there is no detectable reduction in activity). The basal activity is the level of GTP binding observed in the assay in the absence of added histamine or any other agonist, and in the further absence of any test compound. Neutral antagonists of H3 receptor may, but need not, inhibit the binding of agonist to H3 receptor.

As used herein a “H3 receptor agonist” is a compound that elevates the activity of the receptor above the basal activity level of the receptor. H3 receptor agonist activity may be identified using the representative assay provided in Example 7. In general, such an agonist has an EC₅₀ value of less than 4 micromolar, preferably less than 1 micromolar, 500 nanomolar, 100 nanomolar, 50 nanomolar or 10 nanomolar within the assay provided in Example 7. If the GTP binding activity brought about by a test compound attains the same level to that of histamine, it is defined as a full agonist. If the level of GTP binding activity brought about by a test compound is above baseline but below the level attained by histamine, it is defined as a partial agonist. Preferred antagonists do not elevate GTP binding activity under such conditions more than 10% above baseline, preferably not more than 5% above baseline, and most preferably not more than 2% above baseline.

A “therapeutically effective amount” (or dose) is an amount that, upon administration to a patient, results in a discernible patient benefit (e.g., provides detectable relief from a condition being treated). Such relief may be detected using any appropriate criteria, including alleviation of one or more symptoms characteristic of the condition. A therapeutically effective amount or dose generally results in a concentration of compound in a body fluid (such as blood, plasma, serum, CSF, synovial fluid, lymph, cellular interstitial fluid, tears or urine) that is sufficient to alter H3 receptor GTP binding in vitro. It will be apparent that the discernible patient benefit may be apparent after administration of a single dose, or may become apparent following repeated administration of the therapeutically effective dose according to a predetermined regimen, depending upon the indication for which the compound is administered.

A “patient” is any individual treated with a substituted aryl-fused spirocyclic amine provided herein. Patients include humans, as well as other animals such as companion animals (e.g., dogs and cats) and livestock. Patients may be experiencing one or more symptoms of a condition responsive to H3 receptor modulation, or may be free of such symptom(s) (e.g., treatment may be prophylactic).

### Substituted Aryl-Fused Spirocyclic Amines

As noted above, the present invention provides substituted aryl-fused spirocyclic amines of Formula I:

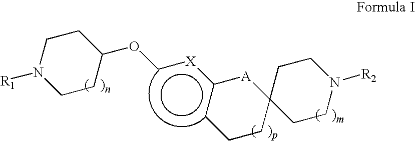

in which variables are as described above.

Within certain aspects, such compounds are H3 receptor modulators that may be used in a variety of contexts, including in the therapeutic treatment of human and animal patients as discussed below. H3 receptor modulators may also be used within in vitro assays (e.g., assays for receptor activity), and as probes for detection and localization of H3 receptor.

As noted above,

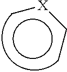

represents an optionally substituted 5- or 6-membered heteroaryl that is fused to the ring represented by

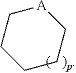

Four of the ring positions are as indicated (i.e., the ring position designated “X” may be CH or N; the ring position linked to the oxygen atom is carbon; and the ring positions that are shared with the fused ring

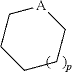

are also carbon). If the ring designated

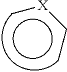

is a six-membered ring, then the remaining ring positions are independently chosen from CH and N. If the ring designated

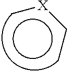

is a five-membered ring, then the remaining ring position is CH, O, S or NR (wherein R is hydrogen or a substituent such as C₁-C₆alkyl or (C₃-C₈cycloalkyl)C₁-C₄alkyl), such that the ring is aromatic.

Certain substituted aryl-fused spirocyclic amines of Formula I further satisfy Formula II, in which the

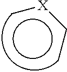

moiety is phenyl, pyridine, pyrimidine, pyrazine, pyridazine or triazine:

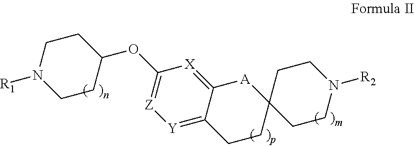

Within Formula H, X, Y and Z are independently CH or N.

Certain substituted aryl-fused spirocyclic amines of Formula II further satisfy Formula IIa or IIb:

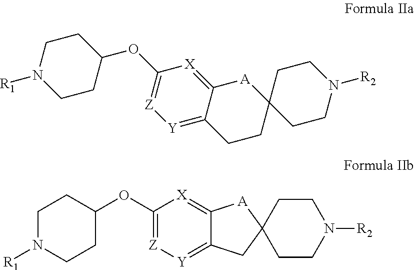

in which variables are as described above.

As noted above, the variables X, Y and Z are each independently CH or N. In certain embodiments, X is N and Y and Z are both CH. In other embodiments, Y is N and X and Z are both CH. In still further embodiments, X, Y and Z are all CH.

Other substituted aryl-fused spirocyclic amines of Formula I further satisfy Formula III:

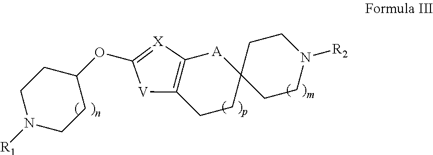

in which V is NR, O or S.

Certain substituted aryl-fused spirocyclic amines of Formula III further satisfy Formula IIIa or IIb:

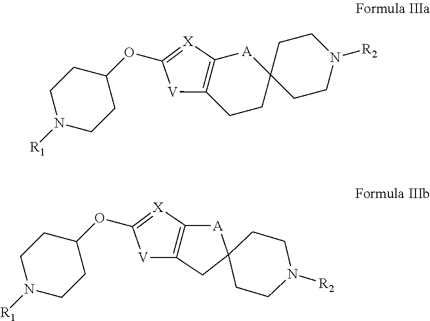

in which variables are as described above.

R₁, in certain compounds provided herein, is cyclopropyl, cyclobutyl, cyclopentyl, cyclohexyl or C₃-C₆alkyl (e.g., propyl, butyl or a branched alkyl group such as isopropyl or tert-butyl).

R₂, in certain compounds provided herein, is phenylC₀-C₂alkyl or (5- or 6-membered heteroaryl)C₀-C₂alkyl, each of which is substituted with from 0 to 4 substituents independently chosen from aminocarbonyl, aminosulfonyl, mono- or di-(C₁-C₆alkyl)aminocarbonyl, mono- or di-(C₁-C₆alkyl)aminosulfonyl, and C₁-C₆alkyl. Representative such R₂ groups include, for example, phenyl, benzyl and pyridyl, each of which is substituted with from 0 to 2 substituents independently chosen from aminocarbonyl, aminosulfonyl, mono- or di-(C₁-C₆alkyl)aminocarbonyl, mono- or di-(C₁-C₆alkyl)aminosulfonyl, and C₁-C₆alkyl.

In other compounds provided herein, R₂ is a group of the formula —W—R₃, wherein W is C(O) or S(O)₂; and R₃ is C₁-C₆alkyl, (C₃-C₈cycloalkyl)C₀-C₂alkyl, mono- or di-(C₁-C₆alkyl)amino, phenyl or 5- or 6-membered heteroaryl, each of which is substituted with from 0 to 4 substituents independently chosen from halogen, hydroxy, amino, aminocarbonyl, aminosulfonyl, C₁-C₆alkyl, C₁-C₆alkoxy, mono- or di-(C₁-C₆alkyDamino, mono- or di-(C₁-C₆alkyl)aminocarbonyl, or mono- or di-(C₁-C₆alkyl)aminosulfonyl. Within certain such compounds, W is C(O); and R₃ is C₁-C₆alkyl, mono- or di-(C₁-C₆alkyl)amino, phenyl or pyridyl, each of which is substituted with from 0 to 2 substituents independently chosen from halogen, hydroxy, amino, aminocarbonyl, aminosulfonyl, C₁-C₆alkyl, C₁-C₆alkoxy, or mono- or di-(C₁-C₆alkyl)amino.

Within still further compounds provided herein, R₂ is C₁-C₆alkyl, (C₃-C₈cycloalkyl)C₀-C₂alkyl or (4- to 7-membered heterocycloalkyl)C₀-C₂alkyl, each of which is substituted with from 0 to 2 substituents independently chosen from halogen, hydroxy, amino, aminocarbonyl, aminosulfonyl, C₁-C₆alkyl, C₁-C₆alkoxy, or mono- or di-(C₁-C₆alkyl)amino.

Representative substituted aryl-fused spirocyclic amines provided herein include, but are not limited to, those specifically described in Examples 1-3. It will be apparent that the specific compounds recited herein are representative only, and are not intended to limit the scope of the present invention. Further, as noted above, all compounds of the present invention may be present as a free acid or base or as a pharmaceutically acceptable salt, solvate or ester.

In certain aspects, substituted aryl-fused spirocyclic amines provided herein are H3 receptor modulators, as determined using an assay for H3 receptor GTP binding. References herein to an “assay for H3 receptor GTP binding” are intended to refer to the in vitro GTP binding assay provided in Example 7, which may be performed in the presence or absence of added agonist. Briefly, to assess H3 receptor agonist-stimulated GTP binding, a H3 receptor preparation is incubated with a H3 receptor agonist (e.g., histamine or an analogue thereof such as R-alpha-methyhistamine), labeled (e.g., ³⁵S) GTP and unlabeled test compound. Within the assays provided herein, the H3 receptor used is preferably mammalian H3 receptor (e.g., human or rat H3 receptor, and preferably human H3 receptor), and more preferably a chimeric human H3 receptor such as a receptor having the sequence provided in SEQ ID NO:8. The H3 receptor may be recombinantly expressed or naturally expressed. The H3 receptor preparation may be, for example, a membrane preparation from cells that recombinantly express H3 receptor. Incubation with a H3 receptor modulator results in a decrease or increase in the amount of label bound to the H3 receptor preparation, relative to the amount of label bound in the absence of the compound.

As noted above, substituted aryl-fused spirocyclic amines that are H3 receptor antagonists are preferred within certain embodiments. When agonist-contacted cells are contacted with a substituted aryl-fused spirocyclic amine that is a H3 receptor antagonist, the response is preferably reduced by at least 20%, more preferably at least 50% and still more preferably at least 80%, as compared to cells that are contacted with the agonist in the absence of the substituted aryl-fused spirocyclic amine. The IC₅₀ for H3 receptor antagonists provided herein is preferably less than 4 micromolar, less than 1 micromolar, less than 500 nM, less than 100 nM, less than 50 nM or less than 10 nM. In certain embodiments, H3 receptor antagonists provided herein exhibit no detectable agonist activity in the assay of Example 7 at a concentration of compound equal to the IC₅₀. Certain preferred antagonists exhibit no detectable agonist activity in the assay at a concentration of the antagonist that is 100-fold higher than the IC₅₀.

In certain embodiments, preferred H3 receptor modulators provided herein are non-sedating. In other words, a dose of H3 receptor modulator that is twice the minimum therapeutically effective dose causes only transient (i.e., lasting for no more than ½ the time that the therapeutic effect lasts) or preferably no statistically significant sedation in an animal model assay of sedation (using the method described by Fitzgerald et al. (1988) Toxicology 49(2-3):433-9). Preferably, a dose that is any of 5, 10, 20, 30, 40, 50, 60, 70, 80, 90 or 100 times the minimum therapeutically effective dose does not produce statistically significant sedation. More preferably, a H3 receptor modulator does not produce sedation at oral doses of less than 140 mg/kg (preferably less than 50 mg/kg, more preferably less than 30 mg/kg).

If desired, H3 receptor modulators provided herein may be evaluated for certain pharmacological properties including, but not limited to, oral bioavailability (preferred compounds are orally bioavailable to an extent allowing for therapeutically effective concentrations of the compound at oral doses of less than 140 mg/kg, preferably less than 50 mg/kg, more preferably less than 30 mg/kg, even more preferably less than 10 mg/kg, and still more preferably less than 1 mg/kg), toxicity (a preferred H3 receptor modulator is nontoxic when a therapeutically effective amount is administered to a subject), side effects (a preferred H3 receptor modulator produces side effects comparable to placebo when a therapeutically effective amount of the compound is administered to a subject), serum protein binding and in vitro and in vivo half-life (a preferred H3 receptor modulator exhibits an in vivo half-life allowing for Q.I.D. dosing, preferably T.I.D. dosing, more preferably B.I.D. dosing, and most preferably once-a-day dosing). In addition, differential penetration of the blood brain barrier may be desirable for certain H3 receptor modulators. Routine assays that are well known in the art may be used to assess these properties, and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays or whole serum binding assays. In vitro half-lives of compounds may be predicted from assays of microsomal half-life as described within Example 8 of PCT Publication Number WO 06/089076.

As noted above, preferred substituted aryl-fused spirocyclic amines are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, or (4) does not cause substantial release of liver enzymes.

As used herein, a compound that does not substantially inhibit cellular ATP production is a compound that satisfies the criteria set forth in Example 9 of PCT Publication Number WO 06/089076. In other words, cells treated as described in Example 9 therein with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more highly preferred embodiments, such doses do not elevate such serum levels of ALT, LDH or AST by more than 75% or 50% over matched controls. Alternatively, a H3 receptor modulator does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the EC₅₀ or IC₅₀ for the compound.

In other embodiments, certain preferred compounds do not substantially inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred H3 receptor modulators do not induce sister chromatid exchange (e.g., in Chinese hamster ovary cells) at such concentrations.

For detection purposes, as discussed in more detail below, substituted aryl-fused spirocyclic amines provided herein may be isotopically-labeled or radiolabeled. For example, one or more atoms may be replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

### Preparation of Substituted Aryl-Fused Spirocyclic Amines

Substituted aryl-fused spirocyclic amines provided herein may generally be prepared using standard synthetic methods. Starting materials illustrated in the schemes and in the examples are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon as appreciated by those skilled in the art. Each variable in the following schemes refers to any group consistent with the description of the substituted aryl-fused spirocyclic amines provided herein.

Certain abbreviations used in the following Schemes and elsewhere herein are:
BOC tent-butyl carboxyl
BOP benzotriazol-1-yl-oxy-tris(dimethylamino)phosphonium hexafluorophosphate
CDCl₃ deuterated chloroform
δ chemical shift
DCM dichloromethane
DEAD diethyl azodicarboxylate
DMF dimethylformamide
DMSO dimethylsulfoxide
Et₂O diethyl ether
EtOAc ethyl acetate
EtOH ethanol
Eq. equivalent(s)
¹H NMR proton nuclear magnetic resonance
HPLC high pressure liquid chromatography
h hour(s)
Hz hertz
LCMS liquid chromatography/mass spectrometry
LDA lithium diisopropylamide
mCPBA meta-chloro-perbenzoic acid
MS mass spectrometry
(M+1) mass+1
Me methyl
MeOH methanol
min minute(s)
NBS N-bromosuccinimde
PG protecting group, such as BOC or a benzyl group
PTLC preparative thin layer chromatography
rt room temperature
TEA triethylamine
THF tetrahydrofuran
TLC thin layer chromatography

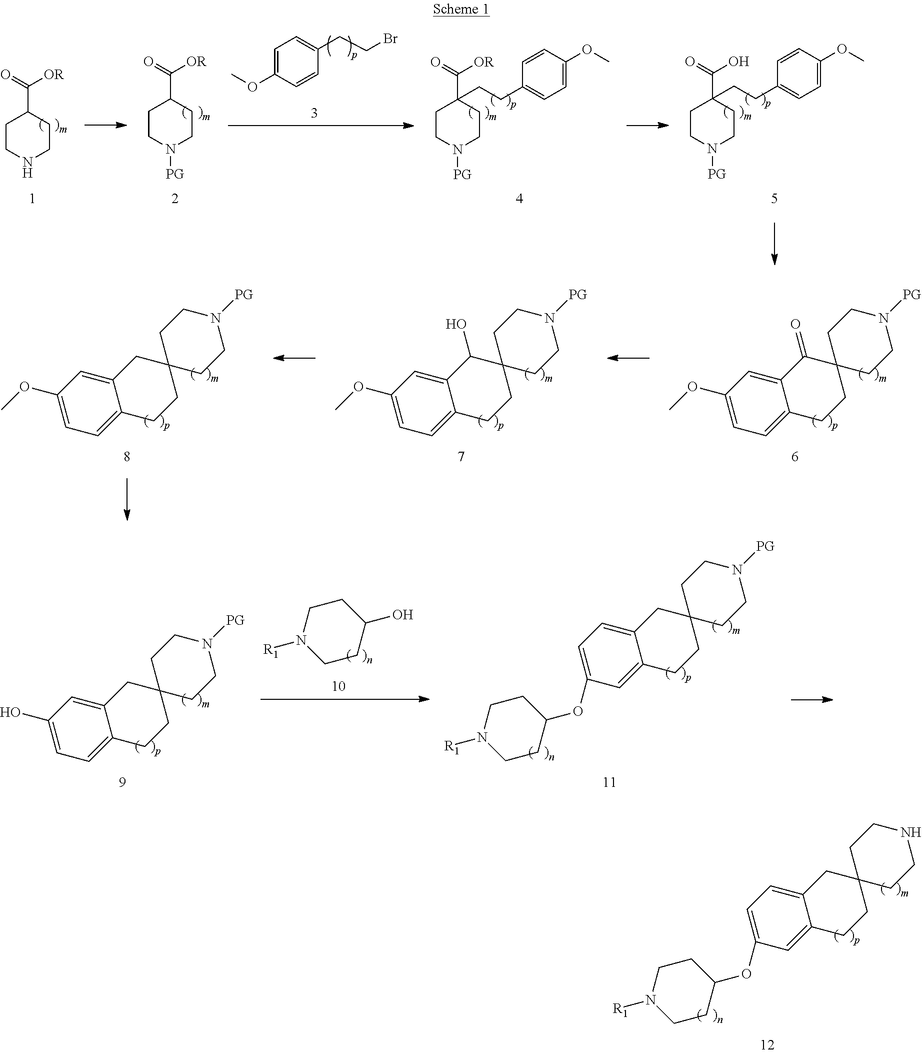

Scheme 1 illustrates the synthesis of compounds of formula 12. Compound 1 (R=Me or Et) is commercially available, known in the literature or conveniently prepared by a variety of methods familiar to those skilled in the art. 1 is reacted with an amine-protecting agent (to insert PG) to give 2. Typical amine-protecting groups include tert-butoxy carbonyl, benzyl and trimethylsilyl. The protected derivative 2 is next reacted with a phenyl alkyl halide, for example 4-methoxyphenylethyl bromide 3 (where q=1), 4-methoxybenzyl bromide (where q=0) or 4-methoxyphenylpropyl bromide (where q=2), in the presence of a strong base such as NaH or LDA, generally in an unreactive solvent such as THF or toluene to afford 4. Hydrolysis of 4 gives 5, which is cyclized by reaction with P₂O₅ to afford 6. Reduction of 6 gives 7, which is further reduced to provide 8. Treatment of 8 with BBr₃ or HBr gives 9, which is reacted with 10 under Mitsunobu condition to afford 11. Compound 10 is commercially available, known in the literature or conveniently prepared by a variety of methods familiar to those skilled in the art. Deprotection of 11 provides 12.

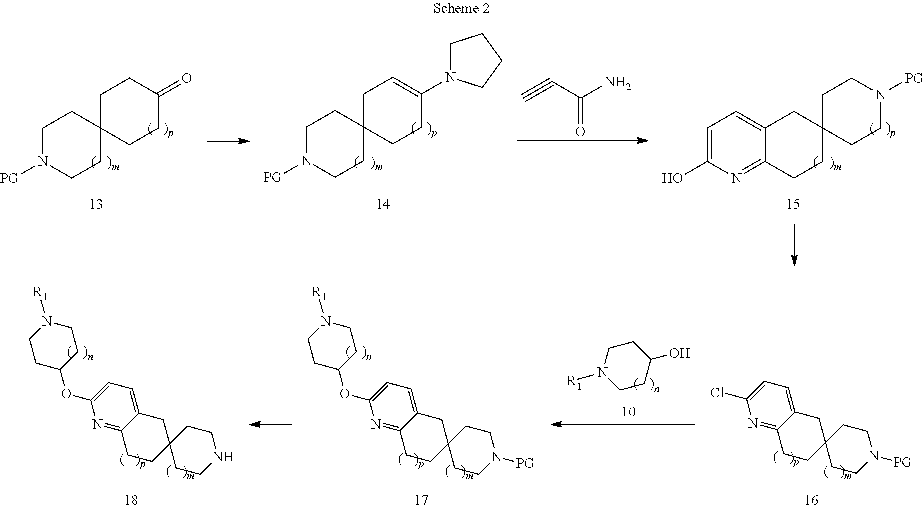

Scheme 2 illustrates the preparation of compounds of formula 18. Compound 13 is commercially available, known in the literature or conveniently prepared by a variety of methods familiar to those skilled in the art. Compound 13 is reacted with pyrrolidine to give 14, which is treated with propiolamide to afford 15. Treatment of 15 with POCl₃ provides 16. The reaction of 16 with 10 in the presence of a base such as NaH or K₂CO₃ gives 17, which is deprotected to afford 18.

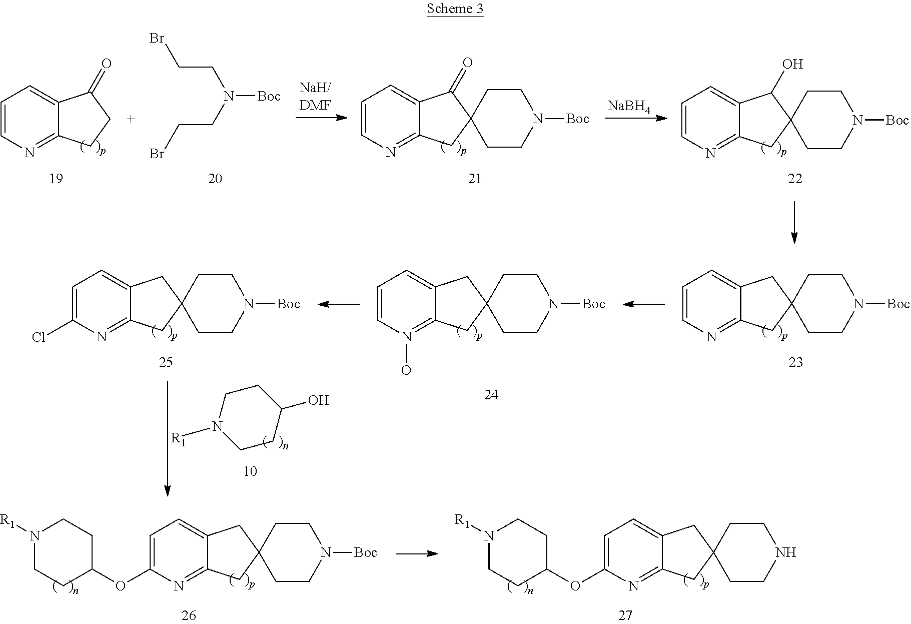

Scheme 3 illustrates the synthesis of compounds of formula 27. Compounds 19 (p=1 or 2) and 20 are commercially available, known in the literature or conveniently prepared by a variety of methods familiar to those skilled in the art. Compound 20 is reacted with 19 in the presence of a strong base such as NaH to provide spiro amine derivative 21. Reduction of 21 gives 22, which is further reduced to give 23. Treatment of 23 with an oxidation reagent such as mCPBA or H₂O₂ provides 24, which is reacted with POCl₃ to afford 25. The reaction of 26 with 10 in the presence of a base such as NaH or K₂CO₃ gives 26, which is deprotected to afford 27.

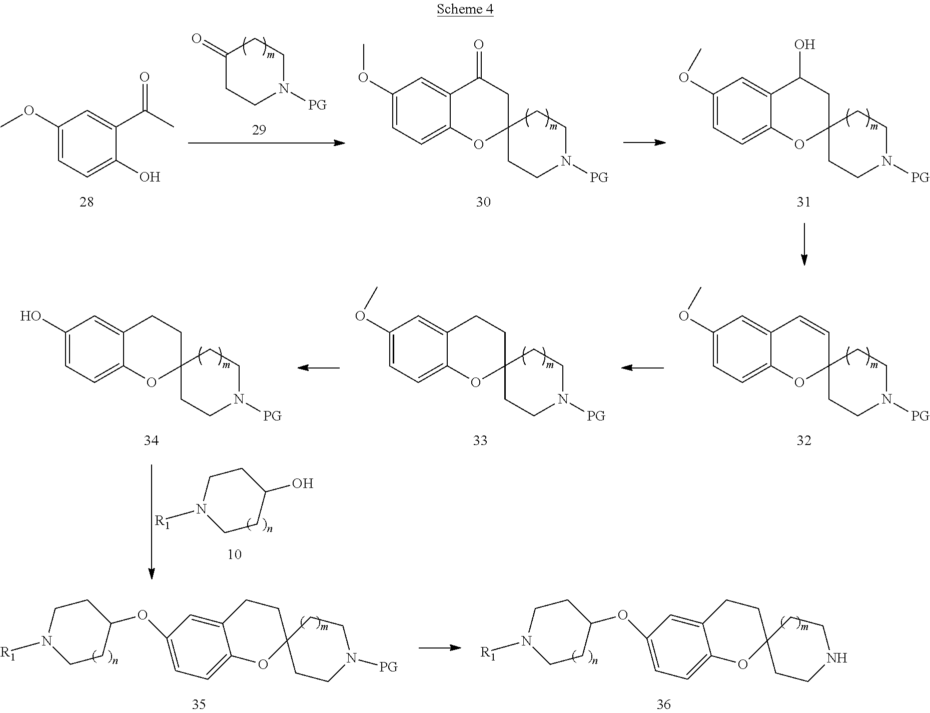

Scheme 4 illustrates the synthesis of compounds of formula 36. Compounds 28 and 29 are commercially available, known in the literature or conveniently prepared by a variety of methods familiar to those skilled in the art. Condensation of 28 with 29 in the presence of a base such as pyrrolidine gives 30, which is reduced to afford 31. Elimination of water of 31 forms 32, which is reduced to give 33. Compound 33 is reacted with BBr₃ or HBr to provide 34, which is reacted with 10 under Mitsunobu condition to afford 35. Deprotection of 35 provides 36.

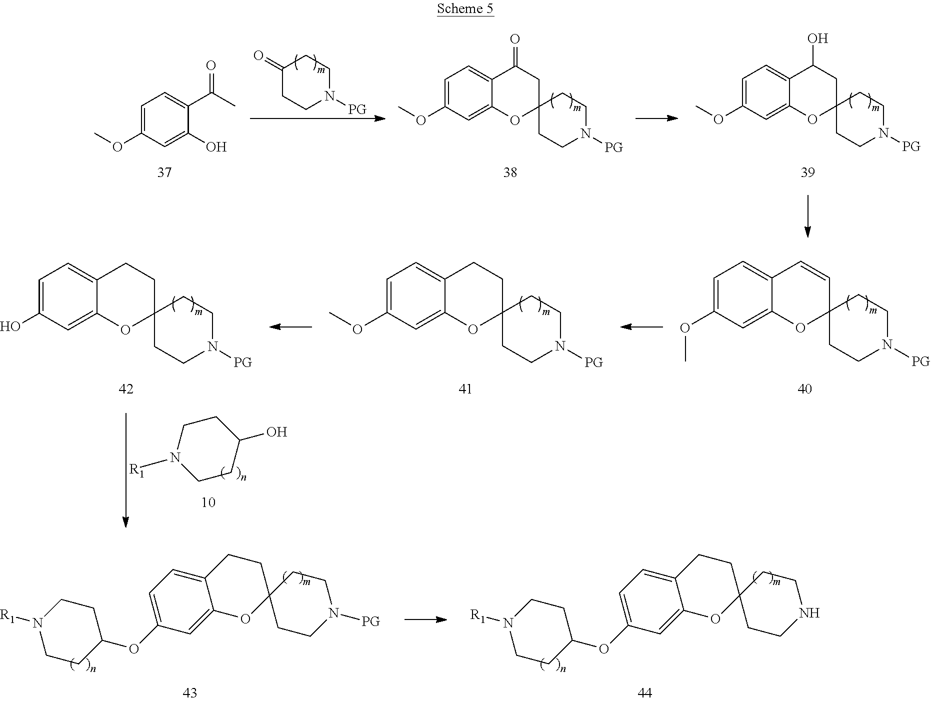

Scheme 5 illustrates the synthesis of compound of formula 44. A synthetic route similar to the one for the preparation of compound 36 (Scheme 4) is used starting from compound 37.

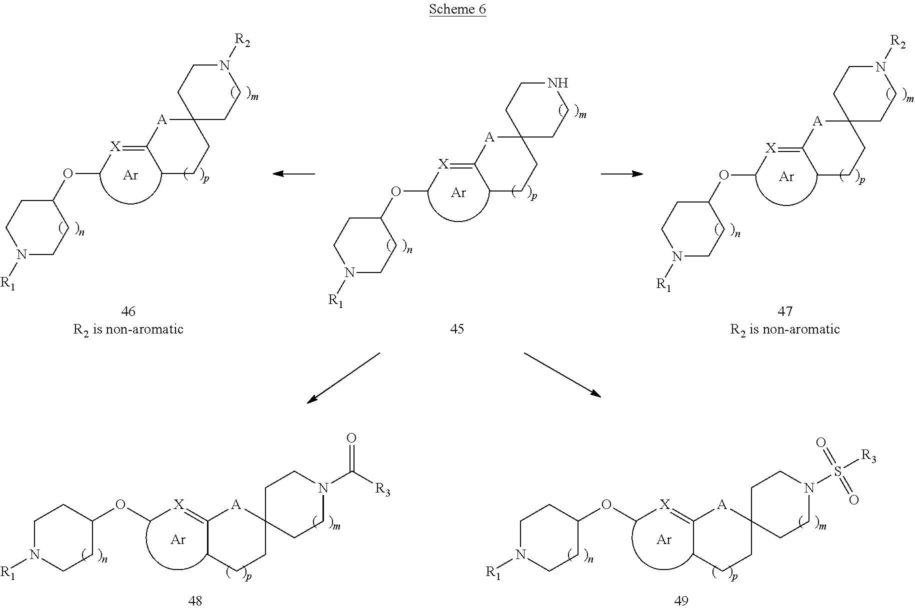

Scheme 6 illustrates the synthesis of compounds 46-49. Compound 45 (representing 12, 18, 27, 36 and 44) is prepared as described in Schemes 1-5; compounds with “Ar” moieties other than those specifically illustrated in Schemes 1-5 may be prepared by analogous procedures. Compound 45 is alkylated with alkyl halide or reacted with aldehyde under reductive amination conditions to give 46. Compound 45 undergoes nucleophilic substitution or Pd-catalyzed coupling reaction with aryl halide (Wagaw and Buchwald (1996) J. Org. Chem. 61:7240) to afford 47. Reaction of 45 with an appropriate acid using a standard coupling agent such as BOP or with an appropriate acid chloride affords 48. Sulfonylation of 45 with an appropriate sulfonyl chloride gives 49.

In certain embodiments, a substituted aryl-fused spirocyclic amine provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Substituted aryl-fused spirocyclic amines may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium-labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

### Pharmaceutical Compositions

The present invention also provides pharmaceutical compositions comprising one or more substituted aryl-fused spirocyclic amine provided herein, together with at least one physiologically acceptable carrier or excipient. Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs or cats). In addition, other active ingredients may (but need not) be included in the pharmaceutical compositions provided herein.

Pharmaceutical compositions may be formulated for any appropriate manner of administration, including, for example, inhalation (e.g., nasal or oral), topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents to increase the bulk weight of the material to be tableted (e.g., calcium carbonate, sodium carbonate, lactose, calcium phosphate or sodium phosphate), granulating and disintegrating agents that modify the disintegration rate in the environment of use (e.g., corn starch, starch derivatives, alginic acid and salts of carboxymethylcellulose), binding agents that impart cohesive qualities to the powdered material(s) (e.g., starch, gelatin, acacia and sugars such as sucrose, glucose, dextrose and lactose) and lubricating agents (e.g., magnesium stearate, calcium stearate, stearic acid or talc). Tablets may be formed using standard techniques, including dry granulation, direct compression and wet granulation. The tablets may be uncoated or they may be coated by known techniques.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active material(s) in admixture with one or more suitable excipients, such as suspending agents (e.g., sodium carboxymethylcellulose, methylcellulose, hydroxypropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, such as ethyl or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be formulated as oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectable aqueous or oleaginous suspension. The active ingredient(s), depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectable compositions, and adjuvants such as local anesthetics, preservatives and/or buffering agents can be dissolved in the vehicle.

Pharmaceutical compositions may also be prepared in the form of suppositories (e.g., for rectal administration). Such compositions can be prepared by mixing the drug with a suitable non-irritating excipient that is solid at ordinary temperatures but liquid at the body temperature and will therefore melt in the body to release the drug. Suitable excipients include, for example, cocoa butter and polyethylene glycols.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated for release at a pre-determined rate. Instantaneous release may be achieved, for example, via sublingual administration (i.e., administration by mouth in such a way that the active ingredient(s) are rapidly absorbed via the blood vessels under the tongue rather than via the digestive tract). Controlled release formulations (i.e., formulations such as a capsule, tablet or coated tablet that slows and/or delays release of active ingredient(s) following administration) may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours. Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, dibutyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed my methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217.

In addition to, or together with, the above modes of administration, a substituted aryl-fused spirocyclic amine provided herein may be conveniently added to food or drinking water (e.g., for administration to non-human animals including companion animals (such as dogs and cats) and livestock) Animal feed and drinking water compositions may be formulated so that the animal takes in an appropriate quantity of the composition along with its diet. It may also be convenient to present the composition as a premix for addition to feed or drinking water.

Substituted aryl-fused spirocyclic amines provided herein are generally present within a pharmaceutical composition at levels providing a therapeutically effective amount upon administration, as described above. Dosage forms providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms generally contain between from about 0.1 mg to about 2 g, preferably 0.5 mg to 1 g, and more preferably 1 mg to 500 mg, of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating conditions responsive to H3 receptor modulation, including those specifically recited herein. Packaged pharmaceutical preparations comprise a container holding one or more dosage units comprising a therapeutically effective amount of at least one H3 receptor modulator as described herein and instructions (e.g., labeling) indicating that the contained composition is to be used for treating a condition responsive to H3 receptor modulation in the patient.

### Methods of Use

H3 receptor modulators provided herein may be used to alter activity and/or activation of H3 receptors in a variety of contexts, both in vitro and in vivo. Within certain aspects, H3 receptor modulators may be used to inhibit or enhance (preferably to inhibit) H3 receptor activity in vitro or in vivo. In general, such methods comprise the step of contacting a H3 receptor with one or more H3 receptor modulators provided herein, in aqueous solution and under conditions otherwise suitable for binding of the modulator(s) to H3 receptor. The H3 receptor modulator(s) are generally present at a concentration that is sufficient to alter H3 receptor GTP binding activity in vitro (using the assay provided in Example 7). The H3 receptor may be present in solution or suspension (e.g., in an isolated membrane or cell preparation), or in a cultured or isolated cell. Within certain embodiments, the H3 receptor is present in a patient (e.g., expressed by a neuronal cell), and the aqueous solution is a body fluid. Preferably, one or more H3 receptor modulators are administered to a patient in an amount such that each H3 receptor modulator is present in at least one body fluid of the patient at a therapeutically effective concentration that is 1 micromolar or less; preferably 500 nanomolar or less; more preferably 100 nanomolar or less, 50 nanomolar or less, 20 nanomolar or less, or 10 nanomolar or less. For example, such compounds may be administered at a dose that is less than 20 mg/kg body weight, preferably less than 5 mg/kg and, in some instances, less than 1 mg/kg. In vivo, modulation of H3 receptor activity may be assessed by detecting an alteration of a symptom (e.g., memory or attention) in a patient being treated with one or more H3 receptor modulators provided herein.

The present invention further provides methods for treating conditions responsive to H3 receptor modulation. Within the context of the present invention, the term “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). A condition is “responsive to H3 receptor modulation” if it is characterized by inappropriate activity of H3 receptor, regardless of the amount of H3 receptor ligand present locally, and/or if modulation of H3 receptor activity results in alleviation of the condition or a symptom thereof. Such conditions may be diagnosed and monitored using criteria that have been established in the art. Patients may include humans, domesticated companion animals and livestock, with dosages as described above.

Conditions that are responsive to H3 receptor modulation include, for example:
Cardiovascular disorders, including atherosclerosis, hypertension, myocardial infarction, coronary heart disease and stroke;
Cancer (e.g., endometrial, breast, prostate and colon cancer, cutaneous carcinoma, medullary thyroid carcinoma and melanoma);
Metabolic disorders including impaired glucose tolerance, dyslipidaemia, and diabetes (e.g., non-insulin dependent diabetes mellitus);
Immune conditions and disorders including osteoarthritis, allergy (e.g., allergic rhinitis), and inflammation;
Respiratory conditions including nasal congestion, upper airway allergic response, asthma and chronic obstructive pulmonary disease;
Disorders associated with the regulation of sleep and wakefulness, or arousal and vigilance, including excessive daytime sleepiness (EDS), shift work disorder, narcolepsy, jet lag, and sleep disorders such as primary insomnia, idiopathic hypersomnia, circadian rhythm sleep disorder, dyssomnia NOS, parasomnias including nightmare disorder, sleep terror disorder, sleep disorders secondary to depression, anxiety and/or other mental disorders and substance-induced sleep disorder;
Fatigue and fatigue-related disorders such as sleep/fatigue disorders, sleep impairment due to perimenopausal hormonal shifts, Parkinson's-related fatigue, multiple sclerosis-related fatigue, and chemotherapy-induced fatigue;
Eating disorders (e.g., bulimia, binge eating and anorexia) and obesity;
Digestive system and gastrointestinal disorders including gallbladder disease, ulcer, hyper- and hypo-motility of the gastrointestinal tract and irritable bowel syndrome;
CNS disorders including hyper- and hypo-activity of the central nervous system, migraine, epilepsy, seizures, convulsions, mood disorders, attention deficit disorder, attention deficit hyperactivity disorder, bipolar disorder, depression, manic disorders, obsessive compulsive disorder, schizophrenia, migraine, vertigo, motion sickness, dementia, cognitive deficit (e.g., in psychiatric disorder, such as mild cognitive impairment), learning deficit, memory deficit (e.g., age-related memory dysfunction), multiple sclerosis, Parkinson's disease, Alzheimer's disease and other neurodegenerative disorders, addiction (e.g., resulting from drug abuse), neurogenic inflammation and Tourette's syndrome;
Vestibular dysfunction (e.g., Meniere's disease, dizziness and motion sickness);
Pain (e.g., inflammatory pain or neuropathic pain) and itch;
Septic shock; and
Glaucoma.

H3 receptor modulators may further be used to enhance a patient's cognitive ability.

In certain embodiments, substituted aryl-fused spirocyclic amines provided herein are used to treat Alzheimer's disease, Parkinson's disease, schizophrenia, mood and attention alterations including attention deficit hyperactivity disorder and attention deficit disorder, memory and learning disorders, cognitive disorders (such as mild cognitive impairment and cognitive deficits in psychiatric pathologies), epilepsy, migraine, and disorders associated with the regulation of sleep and wakefulness, as well as in the treatment and prevention of conditions such as obesity, eating disorders, diabetes, vertigo, motion sickness and allergic rhinitis. Treatment regimens may vary depending on the compound used and the particular condition to be treated. However, for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, and rate of excretion, drug combination and the severity of the particular disease undergoing therapy. In general, the use of the minimum dose sufficient to provide effective therapy is preferred. Patients may generally be monitored for therapeutic effectiveness using medical or veterinary criteria suitable for the condition being treated or prevented.

Within other aspects, H3 receptor modulators provided herein may be used within combination therapy for the treatment of conditions that are responsive to H3 receptor modulation, as described above, or for prevention of drowsiness that might otherwise be induced by pharmacotherapy with another therapeutic agent, such as a dopamine agonist, an anxiolytic, an antidepressant, an antiepilectic agent, an antihypertensive agent, an analgesic, an anti-diabetic agent, or an antipsychotic agent. Within such combination therapy, a H3 receptor modulator is administered to a patient along with a second therapeutic agent that is not a H3 receptor modulator. The H3 receptor modulator and second therapeutic agent may be present in the same pharmaceutical composition, or may be administered separately in either order. It will be apparent that additional therapeutic agents may, but need not, also be administered.

Second therapeutic agents suitable for use in such combination therapy include, for example, antiobesity agents, antidiabetics, antihypertensive agents, antidepressants, antipsychotic agents, anti-inflammatory agents, and agents (including those already mentioned) that produce drowsiness as an unwanted side effect. In certain combinations, the second therapeutic agent is a compound for the treatment of attention deficit disorder or attention deficit hyperactivity disorder, an antipsychotic agent or an anti-obesity agent.

Histamine H1 receptor modulators represent one class of second therapeutic agents. Many H1 receptor modulators (and H2 receptor modulators, such as cimetidine, famotidine, and ranitidine) cause drowsiness. Combination with H1 receptor modulators may be used, for example, in the treatment of Alzheimer's disease, inflammatory diseases and allergic conditions. Representative H1 receptor antagonists include, for example, loratadine, desloratadine, fexofenadine and cetirizine Other H1 receptor antagonists, more commonly associated with drowsiness, include ebastine, mizolastine, acrivastine, astemizole, azatadine, azelastine, brompheniramine, chlorpheniramine, clemastine, cyproheptadine, dexchlorpheniramine, diphenhydramine, hydroxyzine, levocabastine, promethazine and tripelenamine.

Antiobesity therapeutic agents for use in combination therapy include, for example, leptin, leptin receptor agonists, melanin concentrating hormone (MCH) receptor antagonists, melanocortin receptor 3 (MC3) agonists, melanocortin receptor 4 (MC4) agonists, melanocyte stimulating hormone (MSH) agonists, cocaine and amphetamine regulated transcript (CART) agonists, dipeptidyl aminopeptidase inhibitors, a growth hormone secretagogue, beta-3 adrenergic agonists, 5HT-2 agonists, orexin antagonists, neuropeptide Y₁ or Y₅ antagonists, tumor necrosis factor (TNF) agonists, galanin antagonists, urocortin agonists, cholecystokinin (CCK) agonists, GLP-1 agonists, serotonin (5HT) agonists, bombesin agonists, CB1 antagonists such as rimonabant, growth hormone, growth factors such as prolactin or placental lactogen, growth hormone releasing compounds, thyrotropin (TRH) agonists, uncoupling protein 2 or 3 (UCP 2 or 3) modulators, dopamine agonists (e.g., the partial D2 agonist aplindore), agents that modify lipid metabolism such as antilipidemic agents (e.g., cholestyramine, colestipol, clofibrate, gemfibrozil, lovastatin, pravastatin, simvastatin, probucol or dextrothyroxine), lipase/amylase inhibitors, peroxisome proliferator-activated receptor (PPAR) modulators, retinoid X receptor (RXR) modulators, TR-beta agonists, agouti-related protein (AGRP) inhibitors, opioid antagonists such as naltrexone, exendin-4, GLP-1, ciliary neurotrophic factor, corticotropin-releasing factor binding protein (CRF BP) antagonists and/or corticotropin-releasing factor (CRF) agonists. Representative such agents include, for example, sibutramine, dexfenfluramine, dextroamphetamine, amphetamine, orlistat, mazindol, phentermine, phendimetrazine, diethylpropion, fluoxetine, bupropion, topiramate and ecopipam.

Antihypertensive therapeutic agents for use in combination therapy include, for example, beta-blockers such as alprenolol, atenolol, timolol, pindolol, propranolol and metoprolol, angiotensin converting enzyme (ACE) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, alpha-blockers such as doxazocin, urapidil, prazosin and terazosin, and angiotensin receptor blockers such as losartan.

CNS-active agents for use in combination therapy include, but are not limited to the following: for anxiety, depression, mood disorders or schizophrenia: serotonin receptor (e.g., 5-HT_{1A}) agonists and antagonists, neurokinin receptor antagonists, GABAergic agents, and corticotropin releasing factor receptor (CRF₁) antagonists; for sleep disorders: melatonin receptor agonists; and for neurodegenerative disorders (such as Alzheimer's dementia): nicotinic agonists, muscarinic agents, acetylcholinesterase inhibitors and dopamine receptor agonists. For example, such combination therapy may include a selective serotonin reuptake inhibitor (SSRI) or a non-selective serotonin, dopamine and/or norepinephrine reuptake inhibitor. Such agents include, for example, fluoxetine, sertraline, paroxetine, amitriptyline, seroxat and citalopram. For cognitive disorders, representative agents for use in combination therapy include GABAergic agents. Other anti-depressants include amoxapine, bupropion, clomipramine, desipramine, doxepin, duloxetine, escitalopram, fluvoxamine, imipramine, isocarboxazid, maprotiline, mirtazapine, nefazodone, nortriptyline, phenelzine, protriptyline, tranylcypromine, trazodone, trimipramine, and venlafaxine.

Other therapeutic agents suitable for combination therapy include, for example, agents that modify cholinergic transmission (e.g., 5-HT₆ antagonists), M1 muscarinic agonists, M2 muscarinic antagonists and acetylcholinesterase inhibitors.

Suitable doses for H3 receptor modulator within such combination therapy are generally as described above. Doses and methods of administration of other therapeutic agents can be found, for example, in the manufacturer's instructions in the Physician's Desk Reference. In certain embodiments, the combination administration of a H3 receptor modulator with the second therapeutic agent results in a reduction of the dosage of the second therapeutic agent required to produce a therapeutic effect (i.e., a decrease in the minimum therapeutically effective amount). Thus, preferably, the dosage of second therapeutic agent in a combination or combination treatment method is less than the maximum dose advised by the manufacturer for administration of the second therapeutic agent without combination administration of a H3 receptor modulator. More preferably this dosage is less than ¾, even more preferably less than ½, and highly preferably, less than ¼ of the maximum dose, while most preferably the dose is less than 10% of the maximum dose advised by the manufacturer for the second therapeutic agent when administered without combination administration of a H3 receptor modulator. It will be apparent that the dosage amount of H3 receptor modulator component(s) of the combination needed to achieve the desired effect may similarly be affected by the dosage amount and potency of the other therapeutic component(s) of the combination.

In certain preferred embodiments, the combination administration of a H3 receptor modulator with other therapeutic agent(s) is accomplished by packaging one or more H3 receptor modulators and one or more other therapeutic agents in the same package, either in separate containers within the package or in the same contained as a mixture of one or more H3 receptor modulators and one or more other therapeutic agents. Preferred mixtures are formulated for oral administration (e.g., as pills, capsules, tablets or the like). In certain embodiments, the package comprises a label bearing indicia indicating that the one or more H3 receptor modulators and one or more other therapeutic agents are to be taken together for the treatment of attention deficit disorder, attention deficit hyperactivity disorder, schizophrenia, a cognitive disorder (such as mild cognitive impairment), epilepsy, migraine, a sleep disorder, excessive daytime sleepiness (EDS), shift work disorder, narcolepsy, allergic rhinitis, vertigo, motion sickness, a memory disorder such as Alzheimer's disease, Parkinson's disease, obesity, an eating disorder or diabetes.

Within separate aspects, the present invention provides a variety of non-pharmaceutical in vitro and in vivo uses for the substituted aryl-fused spirocyclic amines provided herein. For example, such compounds may be labeled and used as probes for the detection and localization of H3 receptor (in samples such as cell preparations or tissue sections, preparations or fractions thereof). In addition, compounds provided herein that comprise a suitable reactive group (such as an aryl carbonyl, nitro or azide group) may be used in photoaffinity labeling studies of receptor binding sites. Compounds provided herein may further be used as positive controls in assays for receptor activity, as standards for determining the ability of a candidate agent to bind to H3 receptor, or as radiotracers for positron emission tomography (PET) imaging or for single photon emission computerized tomography (SPECT). Such methods can be used to characterize H3 receptors in living subjects. For example, a substituted aryl-fused spirocyclic amine may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound substituted aryl-fused spirocyclic amine is removed (e.g., by washing), and bound substituted aryl-fused spirocyclic amine detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of H3 receptor in the sample. Detection assays, including receptor autoradiography (receptor mapping) of H3 receptor in cultured cells or tissue samples may be performed as described by Kuhar in sections 8.1.1 to 8.1.9 of Current Protocols in Pharmacology (1998) John Wiley & Sons, New York.

Substituted aryl-fused spirocyclic amine provided herein may also be used within a variety of well known cell separation methods. For example, modulators may be linked to the interior surface of a tissue culture plate or other support, for use as affinity ligands for immobilizing and thereby isolating, H3 receptors (e.g., isolating receptor-expressing cells) in vitro. Within one preferred embodiment, a modulator linked to a fluorescent marker, such as fluorescein, is contacted with the cells, which are then analyzed (or isolated) by fluorescence activated cell sorting (FACS).

Substituted aryl-fused spirocyclic amine provided herein may further be used within assays for the identification of other agents that bind to H3 receptor. In general, such assays are standard competition binding assays, in which bound, labeled substituted aryl-fused spirocyclic amine is displaced by a test compound. Briefly, such assays are performed by: (a) contacting H3 receptor with a radiolabeled substituted aryl-fused spirocyclic amine as described herein, under conditions that permit binding of the substituted aryl-fused spirocyclic amine to H3 receptor, thereby generating bound, labeled substituted aryl-fused spirocyclic amine; (b) detecting a signal that corresponds to the amount of bound, labeled substituted aryl-fused spirocyclic amine in the absence of test agent; (c) contacting the bound, labeled substituted aryl-fused spirocyclic amine with a test agent; (d) detecting a signal that corresponds to the amount of bound, labeled substituted aryl-fused spirocyclic amine in the presence of test agent; and (e) detecting a decrease in signal detected in step (d), as compared to the signal detected in step (b), and therefrom identifying an agent that binds to H3 receptor.

The following Examples are offered by way of illustration and not by way of limitation. Unless otherwise specified all reagents and solvent are of standard commercial grade and are used without further purification. Using routine modifications, the starting materials may be varied and additional steps employed to produce other compounds provided herein.

### EXAMPLES

Mass spectroscopy data in this and the following Examples is Electrospray MS, obtained in positive ion mode using a Micromass Time-of-Flight LCT (Micromass, Beverly Mass.), equipped with a Waters 600 pump (Waters Corp., Milford, Mass.), Waters 996 photodiode array detector, Gilson 215 autosampler (Gilson, Inc. Middleton, Wis.), and a Gilson 841 microinjector. MassLynx (Advanced Chemistry Development, Inc; Toronto, Canada) version 4.0 software with OpenLynx processing is used for data collection and analysis. MS conditions are as follows: capillary voltage=3.5 kV; cone voltage=30 V, desolvation and source temperature=350 ° C. and 120 ° C., respectively; mass range=181-750 with a scan time of 0.22 seconds and an interscan delay of 0.05 min.

Sample volume of 1 microliter is injected onto a 50×4.6 mm Chromolith SpeedROD RP-18e column (Merck KGaA, Darmstadt, Germany), and eluted using a 2-phase linear gradient at 6 mL/min flow rate. Sample is detected using total absorbance count over the 220-340 nm UV range. The elution conditions are: Mobile Phase A-95/5/0.05 Water/MeOH/TFA; Mobile Phase B-5/95/0.025 Water/MeOH/TFA. The following gradient is used, with an inject to inject cycle of 2.2 min: 0-0.5 minutes 10-100% B, hold at 100% B to 1.2 minutes, return to 10% B at 1.21 min.

### Example 1

### Preparation of 1′-benzyl-5-[(1-cyclobutylpiperidin-4-yl)oxy]-1,3-dihydrospiro[indene-2,4′-piperidine]

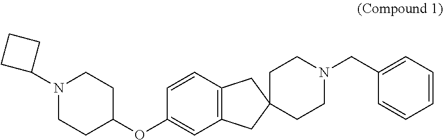

### 1. Ethyl 1-benzylpiperidine-4-carboxylate

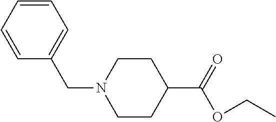

To a mixture of ethyl piperidine-4-carboxylate (50 g) and NaHCO₃ in 50% EtOH (100 mL) at 40° C., benzyl chloride (40.3 g) is added drop wise within one hour. The resulting mixture is stirred at 80° C. for 2 h. The mixture is cooled to rt. Hexane (50 mL) and water (50 mL) are added to the above mixture. The organic layer is separated and washed with water (50 mL). The mixture is dried (Na₂SO₄), and the solvent is evaporated to provide the title compound as an oil, which is used in the next step without purification.

### 2. Ethyl 1-benzyl-4-(4-methoxybenzyl)piperidine-4-carboxylate

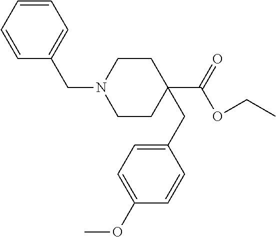

To a solution of ethyl 1-benzylpiperidine-4-carboxylate (4.94 g) in THF (20 mL) at −78° C., LDA (1M in THF) (1.1 eq) is added drop wise over 30 min. The resulting mixture is stirred at −78° C. for one hour. A solution of 4-methoxybenzyl chloride (3.13 g) in THF (10 mL) is added dropwise over 30 min. The mixture is allowed to warm to rt overnight. The reaction is quenched with water (20 mL) and the mixture is extracted with EtOAc (50 mL). The organic layer is separated and dried (Na₂SO₄), and the solvent is removed. Flash column purification of the residue with hexane/ethyl acetate (3:1) provides the title compound as a white solid.

### 3. 1-Benzyl-4-(4-methoxybenzyl)piperidine-4-carboxylic acid

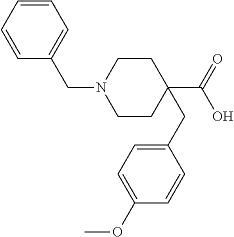

To a solution of ethyl 1-benzyl-4-(4-methoxybenzyl)piperidine-4-carboxylate (5 g) in EtOH (20 mL), is added 20% NaOH solution (2.75 g). The resulting mixture is refluxed overnight. The mixture is cooled to rt and is acidified with concentrated HCl solution to pH around 3. The solid is filtered and dried to give the title product, which is used in the next step without purification.

### 4. 1′-Benzyl-6-methoxyspiro[indene-2,4′-piperidin]-1(3H)-one

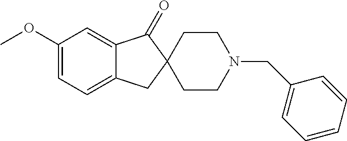

Phosphorus pentoxide (14 g) is dissolved in methanesulfonic acid (150 mL) with stirring at 80-85° C. The crude 1-benzyl-4-(4-methoxybenzyl)piperidine-4-carboxylic acid (12.2 g) is added portion wise over 30 min. The reaction mixture is heated to 110° C. for two hours, cooled to rt, and then poured slowly into ice water. The aqueous mixture is basified with 10 N NaOH and extracted with DCM (100 mL×3). The organic layer is dried (Na₂SO₄), and the solvent is evaporated. Flash column purification of the residue with hexane/EtOAc (3:1) provides the title compound as a white solid.

### 5. 1′-Benzyl-6-methoxy-1,3-dihydrospiro[indene-2,4′-piperidin]-1-ol

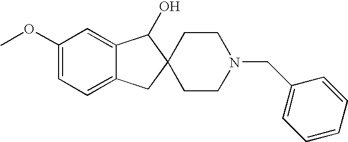

To a solution of 1′-benzyl-6-methoxyspiro[indene-2,4′-piperidin]-1(3H)-one (8 g) in EtOH (20 mL), sodium borohydride (5.6 g) is added portion wise at rt. The mixture is stirred for 1 h and the reaction is quenched with 1 N NaOH (20 mL). The mixture is concentrated under vacuum and the mixture partitioned between EtOAc and water (50 ml/20 ml). After separation of the organic layer, the aqueous phase is extracted with EtOAc (20 mL×2) and the organic phases are combined, washed with brine (20 mL), dried (Na₂SO₄), filtered, and the solvent is evaporated. Flash column purification of the residue with 4% TEA in EtOAc provides the title compound as a white solid.

### 6. 1′-Benzyl-5-methoxy-1,3-dihydrospiro[indene-2,4′-piperidine]

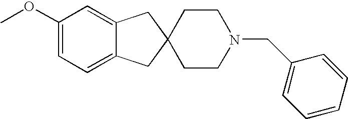

To a solution of 1′-benzyl-6-methoxy-1,3-dihydrospiro[indene-2,4′-piperidin]-1-ol (8 g) in DCM (220 mL), is added trifluoroacetic acid (11 mL) followed by triethylsilane (17.7 mL), and the solution is heated under nitrogen for 12 hours while stirring. The reaction mixture is cooled to rt, is concentrated and partitioned between 2 N NaOH (100 mL) and DCM (150 mL), and the organic phase is separated. The aqueous phase is extracted with DCM (100 mL), and the combined organic phases are washed with brine (100 mL) and dried (Na₂SO₄), and the solvent is evaporated. Flash column purification of the residue with 4% TEA in EtOAc provides the title compound as a white solid.

### 7. 1′-Benzyl-1,3-dihydrospiro[indene-2,4′-piperidin]-5-ol

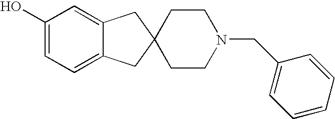

A mixture of 1′-benzyl-5-methoxy-1,3-dihydrospiro[indene-2,4′-piperidine] (3.6 g) in 48% HBr solution (50 mL) in a seal tube is heated at 120° C. overnight. The mixture is cooled to rt and basified with 2 N NaOH to pH around 7. The mixture is extracted with EtOAc (100 mL×2). The extraction is washed with brine, dried (Na₂SO₄), and passed through a silica gel pad. The solvent is evaporated to give the title product.

### 8. tert-Butyl 4-[(1′-benzyl-1,3-dihydrospiro[indene-2,4′-piperidin]-5-yl)oxy]piperidine-1-carboxylate

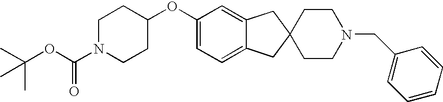

To a solution of 1′-benzyl-1,3-dihydrospiro[indene-2,4′-piperidin]-5-ol (3.3 g) and triphenylphosphine (3.84 g) in THF at 0° C., a solution of tert-butyl 4-hydroxypiperidine-1-carboxylate (2.94 g) and DEAD in THF (60 mL) is added drop wise. The mixture is stirred overnight and the solvent is evaporated. Flash column purification of the residue with hexane/ethyl acetate (3:1) with 1% of TEA provides the title compound.

### 9. 1′-Benzyl-5-(piperidin-4-yloxy)-1,3-dihydrospiro[indene-2,4′-iperidine]

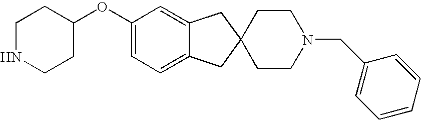

To a solution of tert-butyl 4-[(1′-benzyl-1,3-dihydrospiro[indene-2,4′-piperidin]-5-yl)oxy]piperidine-1-carboxylate (6.1 g) in DCM, trifluoroacetic acid (10 mL) is added. The resulting mixture is stirred at rt for 30 min. The solvent is evaporated and saturated Na₂CO₃ (30 mL) is slowly added. The mixture is extracted with DCM (30 mL×3). The combined organic phase is washed with brine (20 mL), dried (Na₂SO₄), and the solvent is evaporated to give the title product.

### 10. 1′-Benzyl-5-[(1-cyclobutylpiperidin-4-yl)oxy]-1,3-dihydrospiro[indene-2,4′-piperidine]

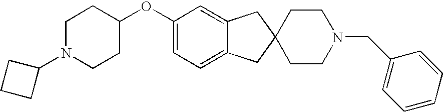

To a suspension of 1′-benzyl-5-(piperidin-4-yloxy)-1,3-dihydrospiro[indene-2,4′-iperidine] (4.5 g) in DCM (50 mL), acetic acid (10 mL) and cyclobutanone (1.2 g) are added. The mixture is stirred at rt for 30 min. NaBH(Ac)₃ (3.6 g) is added portion wise. The resulting mixture is stirred at rt overnight. Saturated Na₂CO₃ solution is added slowly to pH around 8. The organic phase is separated and the aqueous phase is extracted with DCM (30 mL×2). The combined organic phase is washed with brine, dried (Na₂SO₄), and the solvent is evaporated. Flash column purification of the residue with 4% of TEA in EtOAc provides the title compound. MS (M+1) 430.0. ¹H NMR δ 7.32-7.24 (m, 5H), 7.00 (d, 2H), 6.71 (s, 1H), 6.64 (d, 2H), 4.20-4.30 (m, 1H), 3.51 (s, 2H), 2.73-2.58 (m, 6H), 2.48-2.40 (m, 4H), 2.20-1.60 (m, 17H).

### Example 2

### Preparation of Additional Representative Compounds

### A. 5-[(1-CYCLOBUTYLPIPERIDIN-4-YL)OXY]-1′-(4-FLUOROBENZOYL)-1,3-DIHYDROSPIRO[INDENE-2,4′-PIPERIDINE] (COMPOUND 2)

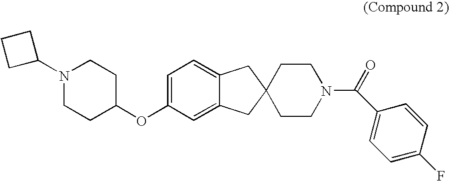

### 1. 5-[(1-Cyclobutylpiperidin-4-yl)oxy]-1,3-dihydrospiro[indene-2,4′-piperidine]

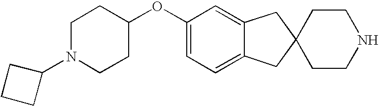

20% Pd(OH)₂ on charcoal (0.5 g) is added to a solution of 1′-benzyl-5-[(1-cyclobutylpiperidin-4-yl)oxy]-1,3-dihydrospiro[indene-2,4′-piperidine] (5.1 g) in EtOH (30 mL). The mixture is hydrogenated at 50 psi overnight. The mixture is filtered through celite, and washed with EtOH. The filtrate is evaporated under reduced pressure to give the title compound. MS (M+1) 341.2.

### 2. 5-[(1-Cyclobutylpiperidin-4-yl)oxy]-1′-(4-fluorobenzoyl)-1,3-dihydrospiro[indene-2,4′-piperidine]

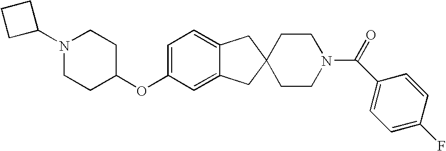

To a solution of 5-[(1-cyclobutylpiperidin-4-yl)oxy]-1,3-dihydrospiro[indene-2,4′-piperidine] (0.2 g) in DCM is added 4-fluorobenzoyl chloride (0.2 g) and TEA (0.2 mL). The resulting mixture is stirred at rt for 1 h. The mixture is washed with saturated NaHCO₃ (10 mL) and brine (10 mL). The DCM solution is dried over Na₂SO₄, and the solvent is removed under reduced pressure to yield a residue which is purified through PTLC (5% TEA in EtOAc) to give the title compound. MS (M+1) 463.2.

### B. 6-{5-[(1-CYCLOBUTYLPIPERIDIN-4-YL)OXY]-1,3-DIHYDRO-1′H-SPIRO[INDENE-2,4′-PIPERIDIN]-1′-YL}-N-METHYLNICOTINAMIDE (COMPOUND 3)

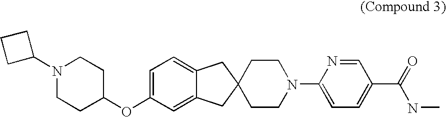

A mixture of 5-[(1-cyclobutylpiperidin-4-yl)oxy]-1,3-dihydrospiro[indene-2,4′-piperidine] (50 mg), 6-chloro-N-methylnicotinamide (37 mg) and K₂CO₃ (30 mg) in DMSO (5 mL) is heated at 120° C. overnight. The mixture is cooled to rt and water (20 mL) is added. The resulting mixture is extracted with DCM (20 mL×2). The combined organic phase is washed with water, dried over Na₂SO₄, and the solvent is removed to give a residue which is purified through PTLC (5% TEA in EtOAc) to give the title compound. MS (M+1) 475.6.

### Example 3

### Preparation of Additional Representative Compounds

Using routine modifications, the starting materials may be varied and additional steps employed to produce other compounds provided herein. Using routine modifications, the starting materials may be varied and additional steps employed to produce other compounds provided herein. Compounds listed in Table I are prepared using such methods. All compound in Table I exhibit a Kᵢ in the assay of Example 7 that is less than 1 micromolar. The molecular weight (presented as M+1) obtained using the method described above is shown in the column headed “MS.”

TABLE I

| | Compound | Name | MS |
|---|---|---|---|
| 4 | 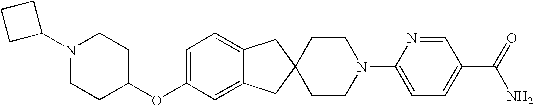 | 6-{5-[(1-cyclobutylpiperidin- 4-yl)oxy]-1,3-dihydro-1′H- spiro[indene-2,4′-piperidin]- 1′-yl}nicotinamide | 461.2 |
| 5 | 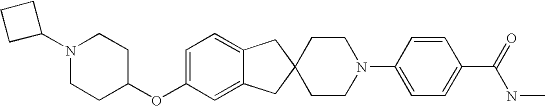 | 4-{5-[(1-cyclobutylpiperidin- 4-yl)oxy]-1,3-dihydro-1′H- spiro[indene-2,4′-piperidin]- 1′-yl}-N-methylbenzamide | 474.3 |
| 6 | 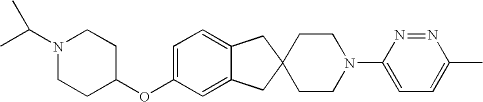 | 5-[(1-cyclobutylpiperidin-4- yl)oxy]-1′-(6-methylpyridazin-3- yl)-1,3-dihydrospiro[indene-2,4′- piperidine] | 433.2 |
| 7 | 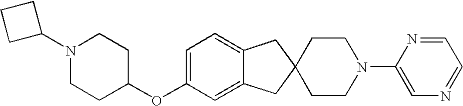 | 5-[(1-cyclobutylpiperidin-4- yl)oxy]-1′-pyrazin-2-yl-1,3- dihydrospiro[indene-2,4′- piperidine] | 419.3 |
| 8 | 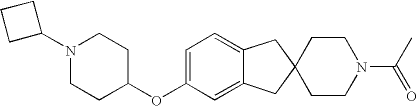 | 1′-acetyl-5-[(1- cyclobutylpiperidin-4-yl)oxy]- 1,3-dihydrospiro[indene-2,4′- piperidine] | 383.2 |
| 9 | 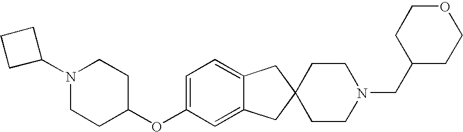 | 5-[(1-cyclobutylpiperidin-4- yl)oxy]-1′-(tetrahydro-2H- pyran-4-ylmethyl)-1,3- dihydrospiro[indene-2,4′- piperidine] | 439.2 |
| 10 | 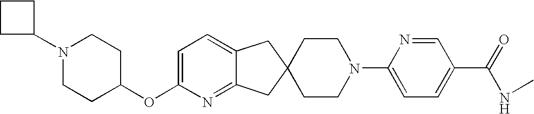 | 6-{2-[(1-cyclobutylpiperidin- 4-yl)oxy]-5,7-dihydro-1′H- spiro[cyclopenta[b]pyridine- 6,4′-piperidin]-1′-yl}-N- methylnicotinamide | 476.2 |
| 11 | 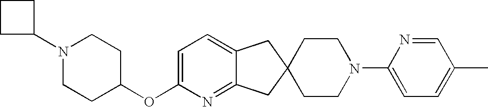 | 2-[(1-cyclobutylpiperidin-4- yl)oxy]-1′-(5-methylpyridin-2- yl)-5,7-dihydrospiro[cyclopenta[b] pyridine-6,4′-piperidine] | 433.2 |
| 12 | 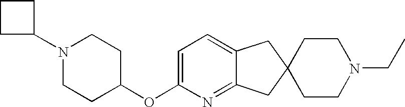 | 2-[(1-cyclobutylpiperidin-4- yl)oxy]-1′-ethyl-5,7- dihydrospiro[cyclopenta[b] pyridine-6,4′-piperidine] | 470.2 |
| 13 | 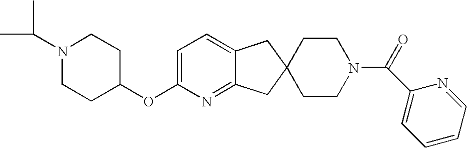 | 2-[(1-isopropylpiperidin-4- yl)oxy]-1′-(pyridin-2- ylcarbonyl)-5,7- dihydrospiro[cyclopenta[b] pyridine-6,4′-piperidine] | 435.2 |
| 14 | 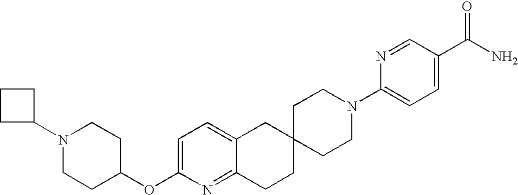 | 6-{2′-[(1-cyclobutylpiperidin-4- yl)oxy]-7′,8′-dihydro-1H,5′H- spiro[piperidine-4,6′- quinolin]-1-yl}nicotinamide | 476.2 |
| 15 | 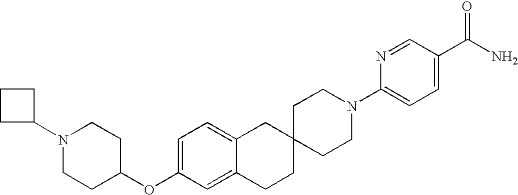 | 6-{6-[(1-cyclobutylpiperidin- 4-yl)oxy]-3,4-dihydro- 1H,1′H-spiro[naphthalene-2,4′- piperidin]-1′-yl}nicotinamide | 475.2 |
| 16 | 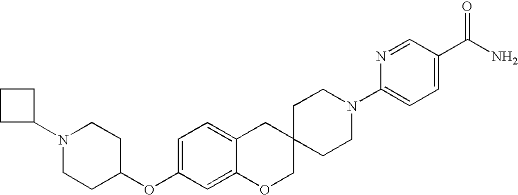 | 6-{7-[(1-cyclobutylpiperidin- 4-yl)oxy]-1′H,4H- spiro[chromene-3,4′-piperidin]- 1′-yl}nicotinamide | 477.2 |
| 17 | 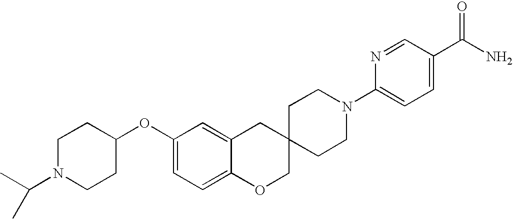 | 6-{6-[(1-isopropylpiperidin- 4-yl)oxy]-1′H,4H- spiro[chromene-3,4′-piperidin]- 1′-yl}nicotinamide | 465.2 |
| 18 | 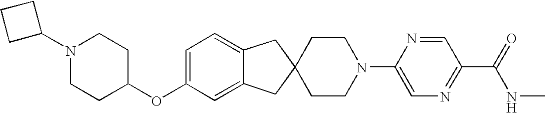 | 5-{5-[(1-cyclobutylpiperidin- 4-yl)oxy]-1,3-dihydro-1′H- spiro[indene-2,4′-piperidin]- 1′-yl}-N-methylpyrazine-2- carboxamide | 476.4 |
| 19 | 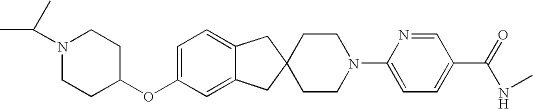 | 6-{5-[(1-isopropylpiperidin-4- yl)oxy]-1,3-dihydro-1′H- spiro[indene-2,4′-piperidin]-1′- yl}-N-methylnicotinamide | |
| 20 | 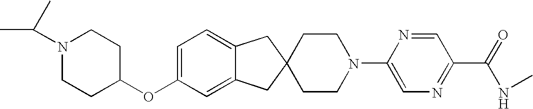 | 5-{5-[(1-isopropylpiperidin-4- yl)oxy]-1,3-dihydro-1′H- spiro[indene-2,4′-piperidin]-1′- yl}-N-methylpyrazine-2- carboxamide | |
| 21 | 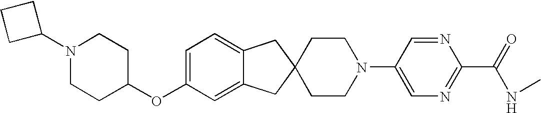 | 5-{5-[(1-cyclobutylpiperidin-4- yl)oxy]-1,3-dihydro-1′H- spiro[indene-2,4′-piperidin]-1′- yl}-N-methylpyrimidine-2- carboxamide | |
| 22 | 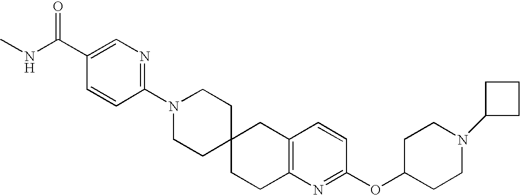 | 6-{2′-[(1-cyclobutylpiperidin-4- yl)oxy]-7′,8′-dihydro-1H,5′H- spiro[piperidine-4,6′-quinolin]-1- yl}-N-methylnicotinamide | |

### Example 4

### Preparation of Chimeric Human H3 Receptor

Chimeric H3 receptor cDNA from human H3 receptor is generated from three cDNA fragments: (1) a human H3 receptor cDNA 5′ fragment; (2) a human H3 receptor cDNA 3′ fragment; and (3) a rat Gαᵢ₂ cDNA fragment, each containing appropriate, overlapping linker sequences, as described in Example 1 of U.S. patent application Ser. No. 11/355,711, which published as US 2006/0188960, and is hereby incorporated by reference for its teaching of the preparation of a chimeric human H3 receptor-rat Gαᵢ₂ baculoviral expression construct that has the sequence provided in SEQ ID NO:7 of US 2006/0188960, and encodes a polypeptide that has the sequence provided in SEQ ID NO:8 of US 2006/0188960.

### Example 5

### Chimeric Human H3 Receptor Baculovirus Preparation and Infection

The chimeric human H3 receptor-rat Gαᵢ₂ baculoviral expression vector is co-transfected along with BACULOGOLD DNA (BD PHARMINGEN, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Kans.) supplemented Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are chosen for a second round of amplification, using 1 ml of passage 1 stock to infect 1×10⁸ cells in 100 ml of insect medium divided into two T175 flasks. Forty-eight hours post infection, passage 2 medium from each 100 ml prep is harvested and plaque assayed to determine virus titer. The cell pellets from the second round of amplification are assayed by affinity binding as described below to verify recombinant receptor expression. A third round of amplification is then initiated using a multiplicity of infection of 0.1 to infect a liter of Sf9 cells. Forty hours post-infection, the supernatant medium is harvested to yield passage 3 baculoviral stock.

The remaining cell pellet is assayed for affinity binding using the protocol of DeMartino et al. (1994) J. Biol. Chem. 269(20):14446-50 (which is incorporated herein by reference for its teaching of binding assays at page 14447), adapted as follows. Radioligand ranges from 0.40-40 nM [³H]—N-(a)methylhistamine (Perkin Elmer, Boston, Mass.) and assay buffer contains 50 mM Tris, 1 mM CaCl₂, 5 mM MgCl₂, 0.1% BSA, 0.1 mM bacitracin, and 100 KIU/ml aprotinin, pH 7.4. Filtration is carried out using GF/C WHATMAN filters (presoaked in 1.0% polyethyeneimine for 2 hr prior to use). Filters are washed three times with 5 ml cold assay buffer without BSA, bacitracin, or aprotinin and air dried for 12-16 hr. Radioactivity retained on filters is measured on a beta scintillation counter.

Titer of the passage 3 baculoviral stock is determined by plaque assay and a multiplicity of infection, incubation time course, binding assay experiment is carried out to determine conditions for optimal receptor expression. A multiplicity of infection of 0.5 and a 72-hr incubation period are preferred infection parameters for chimeric human H3 receptor-rat Gαᵢ₂ expression in up to 1-liter Sf9 cell infection cultures.

Log-phase Sf9 cells (INVITROGEN), are infected with one or more stocks of recombinant baculovirus followed by culturing in insect medium at 27° C. Infections are carried out with virus directing the expression of human H3 receptor-rat Gαᵢ₂ in combination with three G-protein subunit-expression virus stocks: 1) rat Gαᵢ₂ G-protein-encoding virus stock (BIOSIGNAL #V5J008), 2) bovine β1 G-protein-encoding virus stock (BIOSIGNAL #V5H012), and 3) human γ2 G-protein-encoding virus stock (BIOSIGNAL #V6B003), which may be obtained from BIOSIGNAL Inc., Montreal.

The infections are conveniently carried out at a multiplicity of infection of 0.5:1.0:0.5:0.5. At 72 hr post-infection, an aliquot of cell suspension is analyzed for viability by trypan blue dye exclusion. If no blue is detected by visual inspection, the Sf9 cells are harvested via centrifugation (3000 rpm/10 min/4° C.).

### Example 6

### Chimeric Human H3 Receptor Cell Membrane Preparations

Sf9 cell pellets obtained as described in Example 5 are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON PT10-35 homogenizer (KINEMATICA AG, Lucerne, Switzerland; setting 5 for 30 seconds). The homogenate is centrifuged (536×g/10 min at 4° C.) to pellet the nuclei and unbroken cells. The supernatant containing the membranes is decanted to a clean centrifuge tube, centrifuged (48,000×g/30 min, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until used for radioligand binding or functional response assays. The protein concentration of the resulting membrane preparation (hereinafter termed “P2 membranes”) is conveniently measured using a Bradford protein assay (BIO-RAD LABORATORIES, Hercules, Calif.). By this measure, a 1-liter culture of cells typically yields 100-150 mg of total membrane protein.

### Example 7

### Chimeric Human H3 Receptor GTP Binding Assays

This Example illustrates a representative assay for evaluating agonist-stimulated GTP-gamma³⁵S binding (“GTP binding”) activity. Such GTP binding activity can be used to identify H3 antagonists and to differentiate neutral antagonist compounds from those that possess inverse agonist activity. This agonist-stimulated GTP binding activity can also be used to detect partial agonism mediated by antagonist compounds. A compound analyzed in this assay is referred to herein as a “test compound.”

Four independent baculoviral stocks (one directing the expression of the chimeric human H3 receptor and three directing the expression of each of the three subunits of a heterotrimeric G-protein) are used to infect a culture of Sf9 cells as described above. P2 membranes are prepared as described above, and agonist-stimulated GTP binding on the P2 membranes is assessed using histamine (Sigma Chemical Co., St. Louis, Mo.) as agonist in order to ascertain that the receptor/G-protein-alpha-beta-gamma combination(s) yield a functional response as measured by GTP binding. P2 membranes are resuspended by Dounce homogenization (tight pestle) in GTP binding assay buffer (50 mM Tris pH 7.4, 120 mM NaCl, 5 mM MgCl₂, 2 mM EGTA, 1 mg/ml BSA, 0.2 mg/ml bacitracin, 0.02 mg/ml aprotinin, 0.01 mg/ml saponin, 10 μM GDP) and added to assay tubes at a concentration of 35 μg protein/reaction tube. After adding increasing doses of histamine at concentrations ranging from 10⁻¹² M to 10⁻⁵ M, reactions are initiated by the addition of 125 pM GTP-gamma³⁵S (PERKIN ELMER; Boston, Mass.) with a final assay volume of 0.20 ml. In competition experiments, non-radiolabeled test compounds are added to separate reactions at concentrations ranging from 10⁻¹⁰ M to 10⁻⁶ M along with 1 μM histamine to yield a final volume of 0.20 ml.

Neutral antagonists are antagonists that are substantially free of inherent agonist activity, and include those test compounds that reduce the histamine-stimulated GTP binding activity towards, but not below, baseline levels. In contrast, in the absence of added histamine, inverse agonists reduce the GTP binding activity of the receptor-containing membranes below baseline. The elevation of GTP binding activity above baseline by a compound in the absence of added histamine in this assay demonstrates agonist activity.

After a 60-min incubation at room temperature, reactions are terminated by vacuum filtration over WHATMAN GF/C filters (pre-soaked in wash buffer, 0.1% BSA) followed by washing with ice-cold wash buffer (50 mM Tris pH 7.4, 120 mM NaCl). The amount of receptor-bound (and thereby membrane-bound) GTP-gamma³⁵S is determined by measuring the filter-bound radioactivity, preferably by liquid scintillation spectrometry of the washed filters. Non-specific binding is determined in parallel assays including 10 μM unlabeled GTP-gammaS and typically represents less than 5 percent of total binding. Data is expressed as percent above basal (baseline). The results of GTP binding experiments are analyzed using SIGMAPLOT software (SPSS Inc., Chicago, Ill.). IC₅₀ values are calculated by non-linear regression analysis of dose-response curves using Kaleidograph (Synergy Software, Reading, Pa.).

Alternatively the data is analyzed as follows. First, the average bound radioactivity from negative control wells (no agonist) is subtracted from the bound radioactivity detected for each of the other experimental wells. Second, average bound radioactivity is calculated for the positive control wells (agonist wells). Then, percent inhibition for each compound tested is calculated using the equation:

$$Percent Inhibition = 100 - 100 \times [ \frac{Bound radioactivity in Test Wells}{Bound radioactivity in Agonist Wells} ]$$

The % inhibition data is plotted as a function of test compound concentration and test compound IC₅₀ is determined using a linear regression in which x is ln(concentration of test compound) and y is ln(percent inhibition/(100-percent inhibition). Data with a percent inhibition that is greater than 90% or less than 15% are rejected and are not used in the regression. The IC₅₀ is e^{(-intercept/slope)}.

Calculated IC₅₀ values are converted to Kᵢ values by the Cheng-Prusoff correction (Cheng and Prusoff (1973) Biochem. Pharmacol. 22(23):3099-3108). Accordingly, the following equation: Kᵢ=IC₅₀/(1+[L]/EC₅₀) is used, where [L] is the histamine concentration in the GTP binding assay, and EC₅₀ is the concentration of histamine producing a 50% response, as determined by a dose-response analysis using concentrations of histamine ranging from 10⁻¹⁰ M to 10⁻⁶ M.

To assess agonist or inverse agonist activity of a test compound, this assay is performed in the absence of added histamine, and EC₅₀ values are determined by analogous calculations, where the EC₅₀ is the concentration of test compound producing a 50% response.

### Example 8

### Chimeric Human H3 Receptor Screening: GTP Binding Assays

This Example illustrates a representative screening assay for evaluating inhibition of histamine-stimulated GTP-gamma³⁵S binding. Such GTP binding activity can be used to identify H3 antagonists and inverse agonists. A compound analyzed in this assay is referred to herein as a “test compound,” and the initial identification of antagonists and inverse agonists is performed using a test compound concentration of 4 μM.

Four independent baculoviral stocks (one directing the expression of the chimeric human H3 receptor and three directing the expression of each of the three subunits of a heterotrimeric G-protein) are used to infect a culture of SP cells as described above. P2 membranes are prepared as described above, and are resuspended by Dounce homogenization (tight pestle) in GTP binding assay buffer (50 mM Tris pH 7.4, 120 mM NaCl, 5 mM MgCl₂, 2 mM EGTA, 1 mg/ml BSA, 0.2 mg/ml bacitracin, 0.02 mg/ml aprotinin, 0.01 mg/ml saponin, 10 μM GDP) and added to assay tubes at a concentration of 35 μg protein/reaction tube. Non-radiolabeled test compounds are added to separate reactions at a concentration of 4 μM along with 1 μM histamine (agonist). Reactions are initiated by the addition of 125 pM GTP-gamma³⁵S with a final assay volume of 0.20 ml.

After a 60-min incubation at room temperature, reactions are terminated by vacuum filtration over GF/C filters (pre-soaked in 50 mM Tris pH 7.4, 120 mM NaCl plus 0.1% BSA) followed by washing with ice-cold buffer (50 mM Tris pH 7.4, 120 mM NaCl). The amount of receptor-bound (and thereby membrane-bound) GTP-gamma³⁵S is determined by measuring the bound radioactivity, preferably by liquid scintillation spectrometry of the washed filters. Non-specific binding is determined using 10 uM GTP-gammaS and typically represents less than 5 percent of total binding. After subtraction of non-specific binding, data is expressed as percent inhibition of 1 μM histamine signal.

Neutral antagonists are those test compounds that reduce the histamine-stimulated GTP binding activity towards, but not below, baseline levels. In contrast, in the absence of added histamine, inverse agonists reduce the GTP binding activity of the receptor-containing membranes below baseline. Any test compound that elevates GTP binding activity above baseline in the absence of added histamine in this assay is defined as having agonist activity.

What is claimed is:

1. A compound of the Formula: 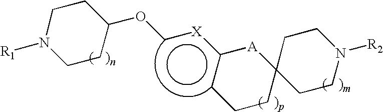
or a pharmaceutically acceptable salt thereof, wherein:
A is CH₂ or O;
X is CH or N, such that 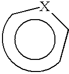
represents a 5- or 6-membered heteroaryl that is fused to the ring represented by 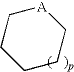
and is optionally substituted with C₁-C₆alkyl or (C₃-C₈cycloalkyl)C₁-C₄alkyl;
n, m and p are independently 0, 1 or 2;
R₁ is C₁-C₆alkyl or (C₃-C₈cycloalkyl)C₀-C₂alkyl, each of which is substituted with from 0 to 4 substituents independently chosen from amino, halogen, cyano, hydroxy, nitro, oxo, aminocarbonyl, aminosulfonyl, —COOH, C₁-C₆alkyl, C₂-C₆alkenyl, C₂-C₆alkynyl, C₁-C₆alkoxy, C₁-C₆haloalkyl, C₁-C₆haloalkoxy, C₂-C₆alkyl ether, C₁-C₆alkanoyl, C₃-C₆alkanone, C₁-C₆alkoxycarbonyl, C₁-C₆alkylsulfonyl, mono- or di-(C₁-C₆alkyl)aminocarbonyl, or mono-or di-(C₁-C₆alkyl)aminoC₀-C₄alkyl;
R₂ is C₁-C₆alkyl, C₂-C₆alkenyl, C₂-C₆alkynyl, (C₃-C₈cycloalkyl)C₀-C₂alkyl, C₁-C₆haloalkyl, C₁-C₆alkanoyl, C₁-C₆alkylsulfonyl, mono- or di-(C₁-C₆alkyl)aminocarbonyl, mono- or di-(C₁-C₆alkyl)aminosulfonyl, phenylC₀-C₂alkyl, (4- to 8-membered heterocycle)C₀-C₂alkyl; each of which is substituted with from 0 to 4 substituents independently chosen from amino, halogen, cyano, hydroxy, nitro, oxo, aminocarbonyl, aminosulfonyl, —COOH, C₁-C₆alkyl, C₂-C₆alkenyl, C₂-C₆alkynyl, C₁-C₆alkoxy, C₁-C₆haloalkoxy, C₂-C₆alkyl ether, C₁-C₆alkanoyl, C₁-C₆alkylsulfonyl, mono- or di-(C₁-C₆alkyl)amino, mono- or di-(C₁-C₆alkyl)aminocarbonyl, mono- or di-(C₁-C₆alkyl)aminosulfonyl, phenyl and 5- or 6-membered heteroaryl.

2. A compound or salt thereof according to claim 1, wherein the compound satisfies the formula: 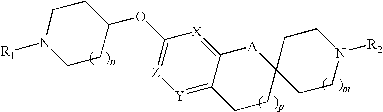
wherein X, Y and Z are independently CH or N.

3. A compound or salt thereof according to claim 2, wherein the compound satisfies the formula:

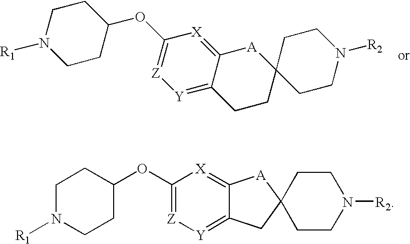

4. A compound or salt thereof according to claim 3, wherein X is N and Y and Z are both CH.

5. A compound or salt thereof according to claim 3, wherein Y is N and X and Z are both CH.

6. A compound or salt thereof according to claim 3, wherein X, Y and Z are all CH.

7. A compound or salt thereof according to claim 3, wherein A is O.

8. A compound or salt thereof according to claim 3, wherein A is CH₂.

9. A compound or salt thereof according to claim 3, wherein R₁ is cyclopropyl, cyclobutyl, cyclopentyl, cyclohexyl or C₃-C₆alkyl.

10. A compound or salt thereof according to claim 9, wherein R₂ is phenylC₀-C₂alkyl or (5- or 6-membered heteroaryl)C₀-C₂alkyl, each of which is substituted with from 0 to 4 substituents independently chosen from aminocarbonyl, aminosulfonyl, mono- or di-(C₁-C₆alkyl)aminocarbonyl, mono- or di-(C₁-C₆alkyl)aminosulfonyl, and C₁-C₆alkyl.

11. A compound or salt thereof according to claim 10, wherein R₂ is phenyl, benzyl or pyridyl, each of which is substituted with from 0 to 2 substituents independently chosen from aminocarbonyl, aminosulfonyl, mono- or di-(C₁-C₆alkyl)aminocarbonyl, mono- or di-(C₁-C₆alkyl)aminosulfonyl, and C₁-C₆alkyl.

12. A compound or salt thereof according to claim 9, wherein R₂ is a group of the formula —W—R₃, wherein:
W is C(O) or S(O)₂; and
R₃ is C₁-C₆alkyl, (C₃-C₈cycloalkyl)C₀-C₂alkyl, mono- or di-(C₁-C₆alkyl)amino, phenyl or 5- or 6-membered heteroaryl, each of which is substituted with from 0 to 4 substituents independently chosen from halogen, hydroxy, amino, aminocarbonyl, aminosulfonyl, C₁-C₆alkyl, C₁-C₆alkoxy, mono- or di-(C₁-C₆alkyl)amino, mono- or di-(C₁-C₆alkyl)aminocarbonyl, or mono- or di-(C₁-C₆alkyl)aminosulfonyl.

13. A compound or salt thereof according to claim 12, wherein:
W is C(O); and
R₃ is C₁-C₆alkyl, mono- or di-(C₁-C₆alkyl)amino, phenyl or pyridyl, each of which is substituted with from 0 to 2 substituents independently chosen from halogen, hydroxy, amino, aminocarbonyl, aminosulfonyl, C₁-C₆alkyl, C₁-C₆alkoxy, or mono- or di-(C₁-C₆alkyl)amino.

14. A compound or salt thereof according to claim 9, wherein R₂ is C₁-C₆alkyl, (C₃-C₈cycloalkyl)C₀-C₂alkyl or (4- to 7-membered heterocycloalkyl)C₀-C₂alkyl, each of which is substituted with from 0 to 2 substituents independently chosen from halogen, hydroxy, amino, aminocarbonyl, aminosulfonyl, C₁-C₆alkyl, C₁-C₆alkoxy, or mono- or di-(C₁-C₆alkyl)amino.

15. A compound or salt thereof according to claim 1, wherein the compound is: 1′-benzyl-5-[(1-cyclobutylpiperidin-4-yl)oxy]-1,3-dihydrospiro[indene-2,4′-piperidine];
5-[(1-Cyclobutylpiperidin-4-yl)oxy]-1′-(4-fluorobenzoyl)-1,3-dihydrospiro[indene-2,4′-piperidine];
6-{5-[(1-Cyclobutylpiperidin-4-yl)oxy]-1,3-dihydro-1′H-spiro[indene-2,4′-piperidin]-1′-yl}-N-methylnicotinamide;
6-{5-[(1-cyclobutylpiperidin-4-yl)oxy]-1,3-dihydro-1′H-spiro[indene-2,4′-piperidin]-1′-yl}nicotinamide;
4-{5-[(1-cyclobutylpiperidin-4-yl)oxy]-1,3-dihydro-1′H-spiro[indene-2,4′-piperidin]-1′-yl}-N-methylbenzamide;
5-[(1-cyclobutylpiperidin-4-yl)oxy]-1′-(6-methylpyridazin-3-yl)-1,3-dihydrospiro[indene-2,4′-piperidine];
5-[(1-cyclobutylpiperidin-4-yl)oxy]-1′-pyrazin-2-yl-1,3-dihydrospiro[indene-2,4′-piperidine];
1′-acetyl-5-[(1-cyclobutylpiperidin-4-yl)oxy]-1,3-dihydrospiro[indene-2,4′-piperidine];
5-[(1-cyclobutylpiperidin-4-yl)oxy]-1′-(tetrahydro-2H-pyran-4-ylmethyl)-1,3-dihydrospiro[indene-2,4′-piperidine];
6-{2-[(1-cyclobutylpiperidin-4-yl)oxy]-5,7-dihydro-1′H-spiro[cyclopenta[b]pyridine-6,4′-piperidin]-1′-yl}-N-methylnicotinamide;
2-[(1-cyclobutylpiperidin-4-yl)oxy]-1′-(5-methylpyridin-2-yl)-5,7-dihydrospiro[cyclopenta[b]pyridine-6,4′-piperidine];
2-[(1-cyclobutylpiperidin-4-yl)oxy]-1′-ethyl-5,7-dihydrospiro[cyclopenta[b]pyridine-6,4′-piperidine];
2-[(1-isopropylpiperidin-4-yl)oxy]-1′-(pyridin-2-ylcarbonyl)-5,7-dihydrospiro[cyclopenta[b]pyridine-6,4′-piperidine];
6-{2′-[(1-cyclobutylpiperidin-4-yl)oxy]-7′,8′-dihydro-1H,5′H-spiro[piperidine-4,6′-quinolin]-1-yl}nicotinamide;
6-{6-[(1-cyclobutylpiperidin-4-yl)oxy]-3,4-dihydro-1H,1′H-spiro[naphthalene-2,4′-piperidin]-1′-yl}nicotinamide;
6-{7-[(1-cyclobutylpiperidin-4-yl)oxy]-1′H,4H-spiro[chromene-3,4′-piperidin]-1′-yl}nicotinamide; or
6-{6-[(1-isopropylpiperidin-4-yl)oxy]-1′H,4H-spiro[chromene-3,4′-piperidin]-1′-yl}nicotinamide.

16-17. (canceled)

18. A pharmaceutical composition, comprising at least one compound or salt according to claim 1 in combination with a physiologically acceptable carrier or excipient.

19. A pharmaceutical composition according to claim 18 wherein the composition is formulated as an injectible fluid, an aerosol, a cream, a gel, a pill, a capsule, a syrup or a transdermal patch.

20-21. (canceled)

22. A method for treating a patient having a condition responsive to H3 receptor modulation, comprising administering to the patient a therapeutically effective amount of a compound or salt of claim 1 to the patient, wherein the condition is attention deficit disorder, attention deficit hyperactivity disorder, dementia, schizophrenia, a cognitive disorder, epilepsy, migraine, excessive daytime sleepiness, shift work sleep disorder, jet lag, fatigue or a fatigue-related disorder, narcolepsy, sleep apnea, allergic rhinitis, vertigo, motion sickness, a memory disorder, obesity, and eating disorder, diabetes, or Parkinson's disease.

23. (canceled)

24. A method according to claim 22, wherein the patient is a human.

25. (canceled)

26. A method for determining the presence or absence of H3 receptor in a sample, comprising the steps of:
(a) contacting a sample with a compound or salt or hydrate thereof according to claim 1, under conditions that permit binding of the compound to H3 receptor; and
(b) detecting a level of the compound bound to H3 receptor, and therefrom determining the presence or absence of H3 receptor in the sample.

27. A method according to claim 26, wherein the compound is radiolabeled, and wherein the step of detection comprises the steps of:
(i) separating unbound compound from bound compound; and
(ii) detecting the presence or absence of bound compound in the sample.

28. A packaged pharmaceutical preparation, comprising:
(a) a pharmaceutical composition according to claim 18 in a container; and
(b) instructions for using the composition to treat a condition responsive to H3 receptor modulation in a patient, wherein the condition is attention deficit disorder, attention deficit disorder, attention deficit hyperactivity disorder, dementia, schizophrenia, a cognitive disorder, epilepsy, migraine, excessive daytime sleepiness, shift work sleep disorder, jet lag, fatigue or a fatigue-related disorder, narcolepsy, sleep apnea, allergic rhinitis, vertigo, motion sickness, a memory disorder, obesity, an eating disorder, diabetes, or Parkinson's disease.

29-33. (canceled)
